(19) United States
(12) Patent Application Publication
(10) Pub. No.: US 2019/0119436 A1
(43) Pub. Date: Apr. 25, 2019

(54) ANTIMICROBIAL POLYMERIC PARTICLES

(71) Applicant: BAR-ILAN RESEARCH & DEVELOPMENT COMPANY LTD, Ramat Gan, IL (US)

(72) Inventors: Shlomo MARGEL, Rehovot (IL); Sarit COHEN, Peduel (IL)

(21) Appl. No.: 16/086,884

(22) PCT Filed: Mar. 28, 2017
(86) PCT No.: PCT/IL2017/050385

§ 371 Date: Sep. 20, 2018

Related U.S. Application Data
(60) Provisional application No. 62/313,837, filed on Mar. 28, 2016.

(51) Int. Cl. C08G 61/04; C08F 212/36; C08F 220/20; C08K 5/14; C08K 5/23; C08K 5/42; C08L 39/06; C08L 53/00; A61L 2/23; C09D 5/16

(52) U.S. Cl. CPC C08G 61/04; C08F 212/36; C08F 220/20; C08K 5/14; C08K 5/23; C09D 165/04; C08L 39/06; C08L 53/005; A61L 2/23; C09D 5/1662; A61L 2202/20; C08K 5/42

(57) ABSTRACT

Disclosed methyl styrene farmin compounds, and crosslinked polymeric backbones comprising same. Further disclosed microsized and nanosized polymeric particles comprising polyisothiouronium methylstyrene (PITMS) and/or poly(methyl styrene farmin) or a derivative thereof, and uses thereof in, for example, reducing or preventing growth of microorganisms.

This application claims the benefit of priority from U.S. Provisional Patent Application No. 62/313,838, filed on Mar. 28, 2016. The content of the above document is incorporated by reference in its entirety as if fully set forth herein.

### FIELD OF THE INVENTION

The present invention, in some embodiments thereof, relates to methyl styrene farmin compounds and derivatives thereof, and to crosslinked polymeric backbones comprising same, and more particularly, but not exclusively, to microsized and nanosized polymeric particles comprising polyisothiouronium methylstyrene (PITMS) and/or poly(methyl styrene farmin) or derivatives thereof, and uses thereof in, for example, reducing or preventing growth of microorganisms.

### BACKGROUND OF THE INVENTION

Bacterial attachment to surfaces leading to the formation of communities of bacterial cells is a major problem in many diverse settings. This sessile community of microorganisms, also termed a biofilm, is attached to an interface, or to each other, and embedded in an exopolymeric matrix. It manifests an altered mode of growth and transcribes genes that free-living microorganisms do not transcribe. The most characteristic phenotype of the biofilm mode of growth is its inherent resistance to disinfection, antimicrobial treatment and immune response killing.

The inherent resistance of biofilms to killing and their pervasive involvement in product contamination, pipe clogging and implant-related infections has prompted for various industrial applications such as drinking water distribution systems and food packaging.

Coating formulations have long included biocidal compounds in an effort to reduce or eliminate microbial growth on the surfaces of these architectural coatings. However, the active life of biocidal compounds in coatings is severely limited by the evaporation or volatilization of these compounds after drying of the coating and unexpected reactions of functional groups within these compounds with other components within the paint formulation.

Micro- and nano-sized particles have many uses in the industrial, commercial, and medicinal arts. These particles can be constructed from polymeric materials, either naturally-occurring or synthetic. Nanoparticles can be crosslinked as well as modified or derivatized by conventional organic chemistry techniques to enhance their use in a variety of technologies.

CN Patent No. 103,044,611 discloses polymeric antibacterial nanoparticles and magnetic antibacterial nanoparticles containing halamine functional groups.

### SUMMARY OF THE INVENTION

The present invention, in some embodiments thereof, relates methyl styrene farmin compounds and derivatives thereof, and to crosslinked polymeric backbones comprising same, and more particularly, but not exclusively, to microsized and nanosized polymeric particles comprising polyisothiouronium methylstyrene (PITMS) and/or poly(methyl styrene farmin) or derivatives thereof, and uses thereof in, for example, reducing or preventing growth of microorganisms.

According to an aspect of some embodiments of the present invention, there is provided a compound being in the form of Formula I:

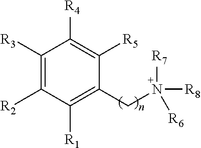

wherein each of R₁-R₈ represents a substituent,

wherein:

one to four substituents from R₁ to R₅, in each instance, comprise or are selected from the group consisting of hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, or is a fused ring;

R₆ to R₈, in each instance, comprises or is selected from the group consisting of hydrogen, alkyl, and cycloalkyl;

n is an integer having a value from 1 to 5, and

at least one of R₁ to R₅ is, independently, in the form represented by Formula II:

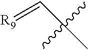

wherein R₉ is alkyl.

In some embodiments, R₃ is in the form represented by Formula II:

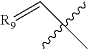

In some embodiments, R₉ is CH₂.

In some embodiments, n is 1.

In some embodiments, the compound is in the form of Formula Ib:

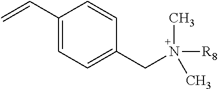

According to an aspect of some embodiments of the present invention, there is provided a polymer comprising a plurality of first monomeric unit, wherein the first monomeric unit is derived from the compound of Formula I.

In some embodiments, the plurality of the first monomeric unit is in the form represented by Formula III:

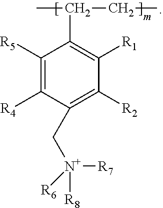

wherein m is an integer having a value from 2 to 3000.

In some embodiments, the disclosed polymer further comprises a plurality of a second monomeric unit, the second monomeric unit being derived from a cross-linking monomer.

In some embodiments, the disclosed polymer comprises a plurality of polymeric backbone represented by Formula IIIc:

Yₐ—Z_{b};

wherein:

Y represents the first monomeric unit;

Z is the second monomeric unit, and

a and b are integers, each independently in each instance, represents the total numbers of Y and Z, respectively, in the polymeric backbone, and

wherein a and b, independently, have a value of 0, 1 or more.

In some embodiments, the second monomeric unit is at least 1%, by weight.

According to an aspect of some embodiments of the present invention, there is provided a polymer comprising a plurality of first monomeric unit derived from a monomer, the first monomeric unit being in the form represented by Formula IV:

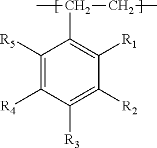

wherein each of R₁ to R₅ represents a substituent,
and wherein one to four substituents from R₁ to R₅, in each instance, comprise or are selected from the group consisting of hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, or is a fused ring, and at least one of R₁ to R₅ is independently in the form represented by Formula V:

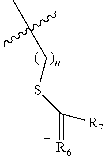

wherein n is an integer having a value from 1 to 5, and

R₆ and R₇ are each, independently selected from the group consisting of NH₂, and

NHR₈, wherein R₈ is or alkyl.

In some embodiments, the first monomeric unit is in the form represented by Formula IVb:

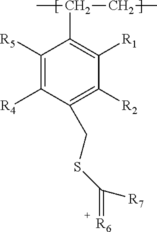

In some embodiments, the first monomeric unit is in the form represented by Formula IVc:

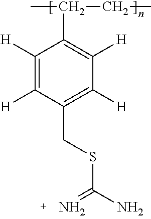

In some embodiments, the polymer, further comprises a plurality of a second monomeric unit derived from a cross-linking monomer.

In some embodiments, the polymer comprises a plurality of polymeric backbone, represented by Formula X:

X_{d}—Fₑ₋;

wherein:

X represents the first monomeric unit;

F represents second monomeric unit derived from a cross-linking monomer;

d and e are integers, each independently in each instance, represents the total numbers of X and F, respectively, in the polymeric backbone, and

wherein d and e, independently, have a value of 0, 1 or more.

In some embodiments, the second monomeric unit is at least 1%, by weight.

In some embodiments, the cross-linking monomer is selected from the group consisting of tetra(ethylene glycol) diacrylate, ethylene glycol dimethacrylate, and divinylbenzene.

According to an aspect of some embodiments of the present invention, there is provided a composition-of-matter comprising the disclosed polymer in any embodiment thereof.

In some embodiments, the composition-of-matter further comprises a stabilizer.

In some embodiments, the stabilizer is selected from the group consisting of polyvinylpyrrolidone (PVP), polysorbate Sodium dodecyl sulfate (SDS), sodium dodecyl benzene sulfate (SDBS), Poly(ethylene glycol)-block-poly(propylene glycol)-block-poly(ethylene glycol), and Polyethylene glycol sorbitan monolaurate (Tween 20).

In some embodiments, the composition-of-matter is in the form of one or more particles.

In some embodiments, the one or more particles are microsized.

In some embodiments, the one or more particles are nanosized.

In some embodiments, at least 80% of the particles are characterized by a size that varies within a range of less than 20%.

In some embodiments, the composition-of-matter in any embodiment thereof further comprises a substrate, wherein a plurality of the polymer is incorporated or coated in/on at least a portion of the substrate.

In some embodiments, the substrate comprises or is made of a polymeric material is selected from the group consisting of polypropylene (PP), polycarbonate (PC), high-density polyethylene (HDPE), polyester (PE), polyethylene terephthalate (PET), polyvinyl chloride (PVC), and polytetrafluoroethylene (PTFE, Teflon®).

In some embodiments, the substrate comprises or is made of a wood, a metal, and glass.

In some embodiments, the substrate is or forms a part of an article.

According to an aspect of some embodiments of the present invention, there is provided an article comprising the disclosed composition-of-matter in any embodiment thereof.

In some embodiments, the article is selected from the group consisting of a medical device, fluidic device, water system device, tubing, an agricultural device, a package, a sealing article, a fuel container and a construction element.

According to an aspect of some embodiments of the present invention, there is provided a method of inhibiting or reducing a formation of load of a microorganism, and/or a formation of a biofilm or biofouling in and/or on an article. In some embodiments the microorganism is selected from the group consisting of: viruses, fungi, parasites, yeast, bacteria, and protozoa.

According to an aspect of some embodiments of the present invention, there is provided a process of producing a particle comprising a polymer, the process comprising the step of free radical homopolymerization or copolymerization of the described monomers in the presence of (a) a free radical initiator; (b) a cross-linking monomer and (c) a stabilizer, thereby making the particle.

In some embodiments, a reaction mixture for making the particle comprises: 1-20% by weight of the monomers of the disclosed monomeric compound;

1-20%, by weight, of the cross-linking monomers;

0.2-20%, by weight, of a free radical initiator; and

0.1-20%, by weight, of the stabilizer.

In some embodiments, the initiator is selected from the group consisting of: azo-bis-isobutyronitrile (AIBN), AIBNCO₂H, potassium persulfate (PPS), benzoyl peroxide, and H₂O₂.

In some embodiments, the cross-linking monomer is selected from the group consisting of tetra(ethylene glycol) diacrylate, ethylene glycol dimethacrylate, and divinylbenzene.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a synthetic scheme of the isothioronium methyl styrene (ITMS) monomer.

FIGS. 2A-B present a SEM image (FIG. 2A) and hydrodynamic size histogram (FIG. 2B) of the Poly (ITMS) (PITMS) microparticles prepared according to some embodiments of the present invention.

FIG. 3 presents a point graph showing the Zeta- (ζ-) potential of the PITMS microparticles at increasing pH values, showing that the isoelectric point is approximately at pH 11, at which the particles are unstable.

FIG. 4 presents a point graph showing the effect of the weight ratio [DVB]/[ITMS+DVB] on the diameter and size distribution of the PITMS microparticles (DVB denotes divinylbenzene).

FIG. 5 presents a point graph showing the effect of the initiator concentration on the diameter and size distribution of the PITMS microparticles.

FIG. 6 presents a point graph showing the effect of polyvinylpyrrolidone (PVP) concentration on the diameter and size distribution of the PITMS particles.

FIG. 7 presents bar graphs showing the antibacterial activity of the PITMS microparticles. The four bacterial strains were grown and treated with either PITMS particles at the indicated concentrations or water (control). The results present the average of three independent experiments.

FIGS. 8A-B present a transmission electron microscopy (TEM) image (FIG. 8A) and a hydrodynamic size histogram (FIG. 8B) of the PITMS nanoparticles NPs.

FIGS. 9A-B present Fourier transform infrared spectroscopy (FTIR) spectra of the ITMS monomer (upper spectrum; FIG. 9A) and the PITMS NPs (lower spectrum; FIG. 9B).

FIGS. 10A-B present X-ray diffraction (XRD) diffraction patterns of the monomer ITMS (upper spectrum; FIG. 10A) and the PITMS NPs (lower spectrum, FIG. 10B).

FIGS. 11A-B present graphs showing the ζ-potential as function of pH (FIG. 11A), and thermo gravimetric analysis (TGA) thermogram of the PITMS NPs (FIG. 11B).

FIG. 12 presents a graph showing the effect of the weight ratio [EGDMA]/[ITMS+EGDMA] on the size and size distribution of the PITMS NPs (EGDMA is ethylene glycol dim ethylacrylate).

FIG. 13 presents a graph showing the effect of the initiator concentration on the size and size distribution of the PITMS NPs.

FIG. 14 presents a graph showing the effect of the total monomer concentration on the diameter and size distribution of the PITMS particles.

FIG. 15 presents a bar graph showing the antibacterial activity of the PITMS NPs against L. innocua. Listeria bacteria were grown and treated with either PITMS NPs at the indicated concentrations or water (control). The results show the pattern observed in at least three independent experiments.

FIG. 16 presents a bar graph showing the biofilm formation of Listeria on polyethylene terephthalate (PET) films.

FIG. 17 presents a bar graph showing the cytotoxic effect of the PITMS NP coatings on HaCaT cells measured by the LDH assay. Cells (3×10⁵) were incubated with the supernatant of the various films that were pre-incubated in the medium according to the experimental section. Cells were incubated with Triton-x-100 1% as positive control (100% toxicity). Untreated cells (negative control) were similarly incubated. Each bar represents mean±SE of 4 separate samples.

FIG. 18 presents a synthetic scheme of the styrene farmin (MSF) monomer. Carbon chain composition in R group (% w) C10: 2% max., C12: 36-44%, C14: 46-54%, C16: 7-13%, C18: 1% max.

FIGS. 19A-D present hydrodynamic diameter histograms (FIGS. 19A, 19B) and TEM images (FIGS. 19C, 19D) of the PMSF NPs prepared by dispersion polymerization in presence (FIGS. 19A, 19C) and in absence (FIGS. 19B, 19D) of PVP, respectively.

FIG. 20 presents FTIR spectra of the MSF (upper panel) and the TTEGDA monomers (middle panel) and the PMSF NPs (lower panel).

FIG. 21 presents a graph showing the ζ-potential of the PMSF NPs as a function of pH.

FIG. 22 presents graphs showing the effect of polymerization time on the hydrodynamic size (graph A) and polymerization yield (graph B) of the formed PMSF NPs.

FIG. 23 presents graphs showing the effect of the total monomers concentration on the size and size distribution of the formed PMSF NPs.

FIG. 24 presents a graph showing the effect of the weight ratio [TTEGDA]/[total monomers] on the size and size distribution of the formed PMSF NPs. (TTEGDA denotes tetra(ethylene glycol) diacrylate).

FIG. 25 presents a graph showing the effect of the initiator concentration on the size and size distribution of the formed PMSF NPs.

FIG. 26 presents a graph showing the effect of the polyvinylpyrrolidone (PVP) concentration on the size and size distribution of the PMSF NPs.

FIG. 27 is a bar graph showing the biofilm formation of Listeria on PET films coated with PMSF NPs.

### DETAILED DESCRIPTION OF THE INVENTION

The present invention, in some embodiments thereof, relates to methyl styrene farmin compounds and to crosslinked polymeric backbones, and more particularly, but not exclusively, to microsized and nanosized polymeric particles comprising poly(isothiouronium methylstyrene) (PITMS) and/or poly(methyl styrene farmin) or a derivative thereof, and uses thereof in, for example, reducing or preventing growth of microorganisms.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The Compound Methyl Styrene Farmin (MSF)

In some embodiments, there is provided herein a compound having the general Formula I:

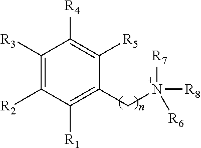

wherein each of R₁-R₇ represents a substituent.

In some embodiments, one to four substituents from R₁ to R₅, in each instance, comprise hydrogen. In some embodiments, one to four substituents from R₁ to R₅, in each instance, comprise alkyl. In some embodiments, one to four substituents from R₁ to R₅, in each instance, comprise cycloalkyl. In some embodiments, one to four substituents from R₁ to R₅, in each instance, comprise aryl. In some embodiments, one to four substituents from R₁ to R₅, in each instance, comprise heteroalicyclic. In some embodiments, one to four substituents from R₁ to R₅, in each instance, comprise heteroaryl. In some embodiments, one to four substituents from R₁ to R₅, in each instance, comprise alkoxy. In some embodiments, one to four substituents from R₁ to R₅, in each instance, comprise hydroxyl. In some embodiments, one to four substituents from R₁ to R₅, in each instance, comprise thiohydroxy. In some embodiments, one to four substituents from R₁ to R₅, in each instance, comprise thioalkoxy. In some embodiments, one to four substituents from R₁ to R₅, in each instance, comprise aryloxy. In some embodiments, one to four substituents from R₁ to R₅, in each instance, comprise thioaryloxy. In some embodiments, one to four substituents from R₁ to R₅, in each instance, comprise amino. In some embodiments, one to four substituents from R₁ to R₅, in each instance, comprise nitro. In some embodiments, one to four substituents from R₁ to R₅, in each instance, comprise halo. In some embodiments, one to four substituents from R₁ to R₅, in each instance, comprise trihalomethyl. In some embodiments, one to four substituents from R₁ to R₅, in each instance, comprise cyano. In some embodiments, one to four substituents from R₁ to R₅, in each instance, comprise amide. In some embodiments, one to four substituents from R₁ to R₅, in each instance, comprise carboxy. In some embodiments, one to four substituents from R₁ to R₅, in each instance, comprise sulfonyl. In some embodiments, one to four substituents from R₁ to R₅, in each instance, comprise sulfoxy. In some embodiments, one to four substituents from R₁ to R₅, in each instance, comprise sulfinyl. In some embodiments, one to four substituents from R₁ to R₅, in each instance, comprise sulfonamide. In some embodiments, one to four substituents from R₁ to R₅, in each instance, comprise or is a fused ring.

Herein, in some embodiments, by “one to four substituents from R₁ to R₅” it is meant refer to: one substituent, two substituents, three substituents, or, in some embodiments to four substituents from R₁ to R₅.

In some embodiments, R₆ to R₈, in each instance, comprises or is alkyl. In some embodiments, R₆ to R₈, in each instance, comprises or is hydrogen. In some embodiments, R₆ to R₈, in each instance, comprises or is cycloalkyl.

In some embodiments, n is an integer having a value from 1 to 5. In some embodiments, n has a value of 1. In some embodiments, n has a value of 2. In some embodiments, n has a value of 3. In some embodiments, n has a value of 4. In some embodiments, n has a value of 5.

In some embodiments, at least one of R₁ to R₅ is, independently, in the form represented by Formula II:

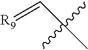

wherein R₉ is alkyl.

In some embodiments, R₉ is CH₂.

In some embodiments, by “at least one of R₁ to R₅” it is meant to refer to one of R₁ to R₅. In some embodiments, by “at least one of R₁ to R₅” it is meant to refer to two of R₁ to R₅. In some embodiments, by “at least one of R₁ to R₅” it is meant to refer to three of R₁ to R₅. In some embodiments, by “at least one of R₁ to R₅” it is meant to refer to four of R₁ to R₅. In some embodiments, by “at least one of R₁ to R₅” it is meant to refer to R₁ to R₅.

In some embodiments, R₃ is in the form represented by Formula II:

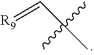

R₉ is defined hereinabove.

In some embodiments, the compound having the general formula I is in the form of Formula Ib (referred to as “MSF”):

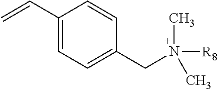

wherein R₈ is defined hereinabove.

In exemplary embodiments, R₈ is H or C10-C18 alkyl.

In some embodiments described herein, any compound and a polymer derived therefrom is in the form of a salt.

In the context of some of the present embodiments, the salt of the hybrid composition described herein may optionally be an acid addition salt comprising at least one group or atom of the composite (e.g., S atom) which is in a positively charged form in combination with at least one counter-ion (e.g., Cl⁻) derived from the selected acid, that forms a salt.

The Poly(MSF):

According to an aspect of some embodiments, there is provided a polymer comprising a plurality of a monomeric unit (also referred to as “first monomeric unit”), wherein the monomeric unit is derived from the monomer (compound) of Formula I in an embodiment thereof.

In some embodiments, the polymer comprises at least two monomeric units.

By “derived from” it is meant to refer to the compound following the polymerization process.

Herein and in the art, the term “monomer” refers to a molecule that may bind chemically to other molecules to form a polymer.

The term “monomeric unit” refers to the repeating structural units, derived from the corresponding monomer.

As used herein, the term “polymer” describes an organic substance composed of a plurality of monomeric units covalently connected to one another.

According to some embodiments, the polymer of the invention can be represented by the general Formula III:

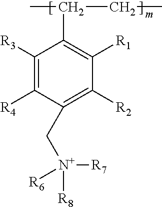

R₁, R₂, R₃, R₄, R₆, R₇, and R₈, are defined hereinabove.

According to some embodiments, the polymer of Formula III can be represented by the general Formula IIIb:

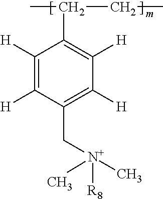

In some embodiments, m is an integer having a value from 1 to 3000. In some embodiments, m is an integer having a value from 1 to 1000. In some embodiments, m is an integer having a value from 1 to 3000. In some embodiments, m is an integer having a value from 1 to 1000. In some embodiments, m is an integer having a value from 2 to 10. In some embodiments, m is an integer having a value from 5 to 20. In some embodiments, m is an integer having a value from 10 to 50. In some embodiments, m is an integer having a value from 20 to 100. In some embodiments, m is an integer having a value from 50 to 200. In some embodiments, m is an integer having a value from 100 to 300. In some embodiments, m is an integer having a value from 200 to 400. In some embodiments, m is an integer having a value from 300 to 500. In some embodiments, m is an integer having a value from 400 to 500.

In some embodiments, n is an integer having a value of 1, 5, 10, 15, 20, 25, 30, 35, 40, 45, 50, 55, 60, 75, 80, 85, 90, 95, 100, 200, 250, 300, 350, 400, 450, or 500, including any value and range therebetween.

In some embodiments, the polymer further comprising a plurality of a second monomeric unit. In some embodiments, the second monomeric unit is derived from a cross-linking monomer (also referred to as: “cross-linker”).

In some embodiments, the polymer further comprising a plurality of a third monomeric unit. In some embodiments, the third monomeric unit is derived from a cross-linking monomer, which is different from the second monomeric unit.

As used herein, “crosslinked” and/or “crosslinking”, and any grammatical derivative thereof, refer generally to a chemical process or the corresponding product thereof in which two chains of polymeric molecules are attached by bridges (crosslinker) composed of an element, a group or a compound, which join certain carbon atoms of the chains by primary chemical.

In some embodiments, the crosslinked polymers have quite different mechanical and physical properties than their uncrosslinked linear or branched counterparts. For example, crosslinked polymers may show unique and highly desirable properties such as solvent resistance, high cohesive strength, and elastomeric character. Typically, the crosslinked polymers are characterized by a plurality of polymeric strands that may be covalently linked together. The term “polymeric strand” refers to any composition of monomeric units covalently bound to define a backbone.

Typically, but not exclusively, the crosslinking reaction can occur in situ during formation of the polymer.

In the context of the present disclosure the cross linker is a compound having at least two double carbon-carbon bonds.

In some embodiments, the polymer comprises a plurality of polymeric backbone represented by Formula IIIc:

Yₐ—Z_{b}—W_{c};

wherein:

Y represents the first monomeric unit;

Z is the second monomeric unit,

W is the third monomeric unit, and

a, b, and c are integers, each independently in each instance, represents the total numbers of Y, Z, and W respectively, in the polymeric backbone.

In some embodiments, a, b, and c independently, have a value of 0, 1 or more.

For example, in some embodiments, a, b, and c, independently, may have a value from 1 to 500. In some embodiments, a, b, and c, independently, may have a value from 1 to 10. In some embodiments, a, b, and c, independently, may have a value from 5 to 20. In some embodiments, a, b, and c, independently, may have a value from 10 to 50. In some embodiments, a, b, and c, independently, may have a value from 20 to 100. In some embodiments, a, b, and c, independently, may have a value from 50 to 200. In some embodiments, a, b, and c, independently, may have a value from 100 to 300. In some embodiments, a, b, and c, independently, may have a value from 200 to 400. In some embodiments, a, b, and c, independently, may have a value from 300 to 500. In some embodiments, a, b, and c, independently, may have a value from 400 to 500.

In some embodiments, a, b, and c, independently, may have a value of 1, 5, 10, 15, 20, 25, 30, 35, 40, 45, 50, 55, 60, 75, 80, 85, 90, 95, 100, 200, 250, 300, 350, 400, 450, and 500, including any value and range therebetween.

In some embodiments, at least two from a, b, and c, have a value above 1.

In some embodiments, W is absent (i.e. c is 0).

In some embodiments, the cross-linking monomer is tetra(ethylene glycol) diacrylate. In some embodiments, the cross-linking monomer is ethylene glycol. In some embodiments, the cross-linking monomer is dimethacrylate.

In some embodiments, the cross-linking monomer is at least 0.5%, by weight of the total polymer. In some embodiments, the cross-linking monomer is at least 0.5%, by weight of the total polymer. In some embodiments, the cross-linking monomer is at least 1%, by weight of the total polymer. In some embodiments, the cross-linking monomer is at least 1.5%, by weight of the total polymer. In some embodiments, the cross-linking monomer is at least 2%, by weight of the total polymer. In some embodiments, the cross-linking monomer is at least 2.5%, by weight of the total polymer. In some embodiments, the cross-linking monomer is at least 3%, by weight of the total polymer. In some embodiments, the cross-linking monomer is at least 3.5%, by weight of the total polymer. In some embodiments, the cross-linking monomer is at least 4%, by weight of the total polymer. In some embodiments, the cross-linking monomer is at least 5%, by weight of the total polymer. In some embodiments, the cross-linking monomer is at least 5.5%, by weight of the total polymer. In some embodiments, the cross-linking monomer is at least 6%, by weight of the total polymer. In some embodiments, the cross-linking monomer is at least 6.5%, by weight of the total polymer. In some embodiments, the cross-linking monomer is at least 7%, by weight of the total polymer. In some embodiments, the cross-linking monomer is at least 7.5%, by weight of the total polymer. In some embodiments, the cross-linking monomer is at least 8%, by weight of the total polymer. In some embodiments, the cross-linking monomer is at least 8.5%, by weight of the total polymer. In some embodiments, the cross-linking monomer is at least 9%, by weight of the total polymer. In some embodiments, the cross-linking monomer is at least 9.5%, by weight of the total polymer. In some embodiments, the cross-linking monomer is at least 10%, by weight of the total polymer.

Polyisothioronium Methylstyrene (PITMS):

According to an aspect of some embodiments, there is provided a polymer comprising a plurality of first monomeric unit derived from a monomer, wherein the first monomeric unit is in the form represented by Formula IV:

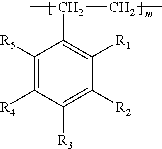

wherein each of R₁ to R₅ represents a substituent, as defined for Formula I hereinabove.

Therefore, in some embodiments, one to four substituents from R₁ to R₅, in each instance, comprise or are selected from the group consisting of hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, or is a fused ring.

In some embodiments, at least one of R₁ to R₅ is independently in the form represented by Formula V:

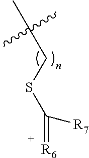

wherein n is an integer.

In some embodiments, n has a value from 1 to 5. In some embodiments, n is an integer having a value of 1. In some embodiments, n is an integer having a value of 2. In some embodiments, n is an integer having a value of 3. In some embodiments, n is an integer having a value of 4. In some embodiments, m is an integer having a value of 5.

In some embodiments, R₆ and R₇ in Formula IV are each, independently selected from the group consisting of NH₂, and NHR₈. In some embodiments, R₈ is alkyl.

In some embodiments, R₆ and R₇ in Formula IV are represented by Formula IVb:

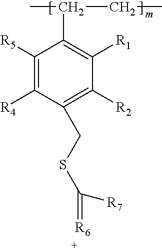

In some embodiments, R₆ and R₇ in Formula IV are both NH₂, e.g., represented by Formula V (referred to as ITMS):

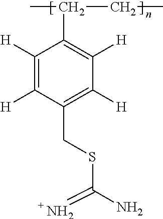

In some embodiments, the polymer comprises a plurality of polymeric backbone represented by Formula VI:

X_{d}—Fₑ-Gₕ;

wherein:

X represents the first monomeric unit (i.e. as represented in Formula IV in any embodiments thereof).

F is the second monomeric unit as defined hereinabove (i.e. derived from a cross-linking monomer), and

G is a third monomeric unit, or is absent (i.e. represented by the Formula VIb: X_{d}—Fₑ);

d, e, and h are integers, each independently in each instance, represents the total numbers of X, F, and G, respectively, in the polymeric backbone.

In some embodiments, d, e, and h are integers, independently, having a value of 0, 1 or more. In some embodiments, at least two integers from d, e, and h, independently, have a value of more than 1.

For example, in some embodiments, d, e, and h, independently, may have a value from 1 to 500. In some embodiments, d, e, and h, independently, may have a value from 1 to 10. In some embodiments, d, e, and h, independently, may have a value from 5 to 20. In some embodiments, d, e, and h, independently, may have a value from 10 to 50. In some embodiments, d, e, and h, independently, may have a value from 20 to 100. In some embodiments, d, e, and h, independently, may have a value from 50 to 200. In some embodiments, d, e, and h, independently, may have a value from 100 to 300. In some embodiments, d, e, and h, independently, may have a value from 200 to 400. In some embodiments, d, e, and h, independently, may have a value from 300 to 500. In some embodiments, d, e, and h, independently, may have a value from 400 to 500.

In some embodiments, d, e, and h, independently, may have a value of 1, 5, 10, 15, 20, 25, 30, 35, 40, 45, 50, 55, 60, 75, 80, 85, 90, 95, 100, 200, 250, 300, 350, 400, 450, and 500, including any value and range therebetween.

In some embodiments, the cross-linking monomer in Formula VI is tetra(ethylene glycol) diacrylate. In some embodiments, the cross-linking monomer is ethylene glycol. In some embodiments, the cross-linking monomer is dimethacrylate.

In some embodiments, the cross-linking monomer in Formula VI is at least 0.5%, by weight of the total polymer. In some embodiments, the cross-linking monomer is at least 0.5%, by weight of the total polymer. In some embodiments, the cross-linking monomer is at least 1%, by weight of the total polymer. In some embodiments, the cross-linking monomer is at least 1.5%, by weight of the total polymer. In some embodiments, the cross-linking monomer is at least 2%, by weight of the total polymer. In some embodiments, the cross-linking monomer is at least 2.5%, by weight of the total polymer. In some embodiments, the cross-linking monomer is at least 3%, by weight of the total polymer. In some embodiments, the cross-linking monomer is at least 3.5%, by weight of the total polymer. In some embodiments, the cross-linking monomer is at least 4%, by weight of the total polymer. In some embodiments, the cross-linking monomer is at least 5%, by weight of the total polymer. In some embodiments, the cross-linking monomer is at least 5.5%, by weight of the total polymer. In some embodiments, the cross-linking monomer is at least 6%, by weight of the total polymer. In some embodiments, the cross-linking monomer is at least 6.5%, by weight of the total polymer. In some embodiments, the cross-linking monomer is at least 7%, by weight of the total polymer. In some embodiments, the cross-linking monomer is at least 7.5%, by weight of the total polymer. In some embodiments, the cross-linking monomer is at least 8%, by weight of the total polymer. In some embodiments, the cross-linking monomer is at least 8.5%, by weight of the total polymer. In some embodiments, the cross-linking monomer is at least 9%, by weight of the total polymer. In some embodiments, the cross-linking monomer is at least 9.5%, by weight of the total polymer. In some embodiments, the cross-linking monomer is at least 10%, by weight of the total polymer.

In some embodiments, the polymer further comprising a plurality of a third monomeric unit. In some embodiments, G, i.e., the third monomeric unit is derived from a cross-linking monomer (also referred to as: “cross-linker”), which is different from the second monomeric unit.

In some embodiments, G represents the first monomeric unit, Y, as defined in Formula IIIc. In exemplary embodiments, there is provided copolymer Poly(MSF-co-ITMS).

In exemplary embodiments, Poly(MSF-co-ITMS) is prepared by dispersion co-polymerization of the monomers MSF and ITMS with a crosslinker monomer such as, without limitation, DVB.

The Compositions-of-Matter:

According to an aspect of some embodiments of the present invention there is provided a composition-of-matter comprising a plurality of any one of the polymer represented by any one of Formulae I to VI, in any embodiment thereof, as described hereinabove, or any combination thereof.

In some embodiments, the composition-of-matter is in the form of one or more particles.

In some embodiments, the composition-of-matter further comprises a stabilizer. In some embodiments, the stabilizer is a surfactant e.g., non-ionic surfactant, anionic surfactant, cationic surfactant and amphiphilic surfactant.

In some embodiments, the stabilizer is selected from, without being limited thereto, Tweens, tritons, tyloxapol, pluronics, Brij cs, Spans, poloxamers and cmulphors.

In some embodiments, the stabilizer is polyvinylpyrrolidone (PVP). In some embodiments, the stabilizer is polysorbate.

In some embodiments, the stabilizer is selected from, but is not limited to, polysorbate Sodium dodecyl sulfate (SDS), sodium dodecyl benzene sulfate (SDBS), Poly(ethylene glycol)-block-poly(propylene glycol)-block-poly(ethylene glycol), and Polyethylene glycol sorbitan monolaurate (Tween 20).

In some embodiments, the particles are microsized. In some embodiments, the particles are nanosized.

In some embodiments, the average or median size (e.g., diameter, length) ranges from about 0.1 micrometer to 10 micrometers. In some embodiments, the average size ranges from about 0.1 micrometer to about 10 micrometers. In some embodiments, the average size ranges from about 0.1 micrometer to about 0.2 micrometers. In some embodiments, the average size ranges from about 0.1 micrometer to about 100 micrometers.

In some embodiments, the average or median size is about 0.1 μm, about 0.2 μm, about 0.3 μm, about 0.4 μm, about 0.5 μm, about 0.6 μm, about 0.7 μm, about 0.8 μm, about 9 μm, or about 10 μm, including any value and size range therebetween.

Hereinthroughout, the terms “nanoparticle”, “nano”, “nanosized”, and any grammatical derivative thereof, which are used herein interchangeably, describe a particle featuring a size of at least one dimension thereof (e.g., diameter, length) that ranges from about 1 nanometer to 100 nanometers. Hereinthroughout NP(s) designates nanoparticle(s).

In some embodiments, the size of the particles described herein represents an average or median size of a plurality of nanoparticle composites or nanoparticles.

In some embodiments, the average or median size of at least e.g., 10%, 20%, 30%, 40%, 50%, 60%, 70%, 80%, 90%, 95% of the polymeric particles, including any value therebetween, ranges from: about 1 nanometer to 100 nanometers, or, in other embodiments, from 10 nm to 100 nm.

In some embodiments, a plurality of the polymeric particles has a uniform size.

By “uniform size” it is meant to refer to diameter size distribution that varies within a range of less than e.g., 60%, 50%, 40%, 30%, 20%, 10%, including any value therebetween.

In some embodiments, plurality of the polymeric particles are characterized by an average hydrodynamic diameter of less than 30 nm with a size distribution of that varies within a range of less than e.g., 60%, 50%, 40,%, 30%, 20%, 10%, including any value therebetween.

In some embodiments, the polymeric particles is about 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 40 nm, about 42 nm, about 44 nm, about 46 nm, about 48 nm, or 50 nm, including any value therebetween.

As used herein the term “average-” or “median-size” refers to diameter of the polymeric particles. The term “diameter” is art-recognized and is used herein to refer to either of the physical diameter (also termed “dry diameter”) or the hydrodynamic diameter. As used herein, the “hydrodynamic diameter” refers to a size determination for the crosslinked polymer in solution (e.g., aqueous solution) using any technique known in the art, e.g., dynamic light scattering (DLS).

In some embodiments, the polymeric particles e.g., PMSF NPS can be dispersed in either aqueous phase or organic phase.

In some embodiments, the stabilizer affects the particle's size, e.g., with the higher concentration of the surfactant, dictating a smaller sized particles.

As exemplified in the Example section that follows, the dry diameter of the polymeric particles, as prepared according to some embodiments of the invention, may be evaluated using transmission electron microscopy (TEM) imaging.

The polymeric particle(s) can be generally shaped as a sphere, a rod, a cylinder, a ribbon, a sponge, and any other shape, or can be in a form of a cluster of any of these shapes, or can comprises a mixture of one or more shapes.

Substrates and/or Articles:

According to some of any of the embodiments described herein, a composition according to any one of the respective embodiments, further comprises a substrate. In some embodiments a plurality of polymeric particles as described in any of the respective embodiments is incorporated in and/or on at least a portion of the substrate.

According to an aspect of some embodiments of the present invention, there is provided a substrate having incorporated in and/or on at least a portion thereof, the disclosed polymeric particles as described herein.

By “a portion thereof” it is meant to refer, for example, to a surface or a portion thereof, and/or a body or a portion thereof, of solid or semi-solid substrates; or a volume or a part thereof, of liquid, gel, foams and other non-solid substrates.

Substrates of widely different chemical nature can be successfully utilized for incorporating (e.g., depositing on a surface thereof) the disclosed polymeric particles thereon, as described herein. By “successfully utilized” it is meant that (i) the disclosed polymeric particles successfully form a uniform and homogenously coating on the substrate's surface (e.g., 1 to 20 microns thickness); and (ii) the resulting coating imparts long-lasting desired properties (e.g., antimicrobial properties) to the substrate's surface.

Substrate usable according to some embodiments of the present invention can therefore be hard (rigid) or soft, solid, semi-solid, or liquid substrates, and may take a form of a foam, a solution, an emulsion, a lotion, a gel, a cream or any mixture thereof.

Substrate usable according to some embodiments of the present invention can have, for example, organic or inorganic surfaces, including, but not limited to, glass surfaces; porcelain surfaces; ceramic surfaces; silicon or organosilicon surfaces, metallic surfaces (e.g., stainless steel); mica, polymeric surfaces such as, for example, plastic surfaces, rubbery surfaces, paper, wood, polymer, a metal, carbon, a biopolymer, silicon mineral (rock or glass), surfaces, wool, silk, cotton, hemp, leather, fur, feather, skin, hide, pelt or pelage) surfaces, plastic surfaces and surfaces comprising or made of polymers such as but not limited to polypropylene (PP), polycarbonate (PC), polyethylene (PET), high-density polyethylene (HDPE), low-density polyethylene (LDPE), polyester (PE), unplasticized polyvinyl chloride (PVC), and fluoropolymers including but not limited to polytetrafluoroethylene (PTFE, Teflon®); or can comprise or be made of any of the foregoing substances, or any mixture thereof.

Alternatively, other portions, or the entire substrate are made of the above-mentioned materials.

The substrate may be coated with the disclosed NPs by any coating method known in the art, e.g., by Mayer rod hand coater. In exemplary embodiments, the NPs (e.g., PMSF NPs) are first dried and then dispersed in organic phase with the substrate (e.g., PET).

In some embodiments, the coating process is assisted by a film former (primer) (e.g., without limitation, G-9/230).

In some embodiments, the substrate incorporating the polymer as described herein is or forms a part of an article.

Hence, according to an aspect of some embodiments of the present invention there is provided an article (e.g., an article-of-manufacturing) comprising a substrate incorporating in and/or on at least a portion thereof a composition-of-matter or the crosslinked polymer, as described in any one of the respective embodiments herein.

The article can be any article which can benefit from the antimicrobial and/or anti-biofilm formation activities of the disclosed polymeric particles.

As further described in the Example section, in some embodiments, the disclosed articles are characterized by desired optical properties (e.g., haze less than 10%, visible light transparency, and clarity).

Exemplary articles include, but are not limited to, medical devices, organic waste processing device, fluidic device, an agricultural device, a package, a sealing article, a fuel container, water and cooling system device and a construction element.

Non-limiting examples of devices which can incorporate the crosslinked polymer, as described herein, beneficially, include medical devices (e.g., catheter), tubing, pumps, drain or waste pipes, screw plates, and the like.

In some embodiments, the article is an element used in water treatment systems (such as for containing and/or transporting and/or treating aqueous media or water), devices, containers, filters, tubes, solutions and gases and the likes.

In some embodiments, the article is an element in a waste treatment system, containers, filters, tubes, and the likes.

Antimicrobial Anti-Biofilm Formation Applications:

While studying the activity of the compositions of matter as described hereinabove, and the activity of compositions-of-matter in polymeric particles are deposited on a substrate's surface, as described herein, the present inventors have surprisingly uncovered that such compositions of matter exhibit high and long lasting antifouling activity and can therefore be beneficially incorporated in articles of in which such an activity is desired. By “long lasting antifouling activity” it is meant to refer to the ability of the compositions-of-matter as described hereinthroughout to withstand repetitive loading cycle of e.g., bacteria. Additionally, or alternatively, it is meant to refer to the ability of the compositions-of-matter as described hereinthroughout to maintain its activity against microbial-based contaminant, within less than 30% variation, up to a period of at least 1 year.

According to some embodiments of the present invention, the composition-of-matter as described hereinthroughout is incorporated within a formulation. In some embodiments, the formulation is used as an antibacterial and/or antifungal cleaner.

According to another aspect of some embodiments of the present invention there is provided a method of inhibiting or reducing or retarding the formation of load of a microorganism and/or the formation of a biofilm, in and/or on an article. The method comprises incorporating in and/or on the article any one of the compositions-of-matter as described herein, including any of the respective embodiments thereof (e.g., comprising a substrate).

Herein “antimicrobial activity” is referred to as an ability to inhibit (prevent), reduce or retard bacterial growth, fungal growth, biofilm formation or eradicate living bacterial cells, or their spores, or fungal cells or viruses in a suspension, on a surface or in a moist environment.

Herein, inhibiting or reducing or retarding the formation of load of a microorganism refers to inhibiting reducing or retarding growth of microorganisms and/or eradicating a portion or all of an existing population of microorganisms.

Thus, the polymeric particles as described herein can be used both in reducing the formation of microorganisms on or in an article, and in killing microorganisms in or on an article or a living tissue.

The microorganism can be, for example, a unicellular microorganism (prokaryotes, archaea, bacteria, eukaryotes, protists, fungi, algae, euglena, protozoan, dinoflagellates, apicomplexa, trypanosomes, amoebae and the likes), or a multicellular microorganism. As used herein, the terms “bacteria”, or “bacterial cells” may refer to either Gram-positive bacteria (e.g., S. aureus, Listeria) and/or Gram-negative bacteria (e.g., E. coli, P. aeruginosa) and/or archae, including multi-drug resistant (MDR) bacteria.

In some embodiments of the present invention the composition-of-matter in any embodiment as described hereinthroughout, may be characterized by high affinity to a specified bacteria type, species, or genus. Therefore, in some embodiments of the present invention the composition-of-matter in any embodiment as described hereinthroughout, the composition-of-matter may be used an effective way for selectively targeting bacteria.

Herein “anti-biofouling activity” or “antifouling activity” is referred to as an ability to inhibit (prevent), reduce or retard biofilm formation on a substrate's surface.

The term “biofilm”, as used herein, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism (prokaryotes, archaea, bacteria, eukaryotes, protists, fungi, algae, euglena, protozoan, dinoflagellates, apicomplexa, trypanosomes, amoebae and the likes), or cells of multicellular organisms in which case the biofilm can be regarded as a colony of cells (like in the case of the unicellular organisms) or as a lower form of a tissue.

In the context of the present embodiments, the cells are of microorganism origins, and the biofilm is a biofilm of microorganisms, such as bacteria and fungi. The cells of a microorganism growing in a biofilm are physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion. The phrases “anti-biofilm formation activity” refers to the capacity of a substance to effect the prevention of formation of a biofilm of bacterial, fungal and/or other cells; and/or to effect a reduction in the rate of buildup of a biofilm of bacterial, fungal and/or other cells, on a surface of a substrate. In some embodiments, the biofilm is formed of bacterial cells (or from a bacterium).

In some embodiments, a biofilm is formed of bacterial cells of bacteria selected from the group consisting of all Gram-positive, Gram-negative bacteria and archaea.

As demonstrated herein, a composition of matter as described herein has shown to exhibit anti-biofilm formation activity and can thus prevent, retard or reduce the formation of a mass of a biofilm.

In some embodiments of the present invention, the activity of preventing or reducing the formation of a biofilm, may be achieved by a substrate or an article incorporating the crosslinked polymer, as described herein.

The inhibition or reduction or retardation of formation of a biofilm assumes that the biofilm has not yet been formed, and hence the presence of the crosslinked polymer nanoparticles is required also in cases where no biofilm is present or detected.

As used herein, the term “preventing” in the context of the formation of a biofilm, indicates that the formation of a biofilm is essentially nullified or is reduced by at least 20%, at least 30%, at least 40%, at least 50%, at least 60%, at least 70%, at least 80%, at least 90%, including any value therebetween, of the appearance of the biofilm in a comparable situation lacking the presence of the disclosed polymeric material or a composition containing same. Alternatively, preventing means a reduction to at least 15%, 10%, or 5% of the appearance of the biofilm in a comparable situation, lacking the presence of the disclosed polymeric material or a composition-of-matter or an article containing same. Methods for determining a level of appearance of a biofilm are known in the art.

As used herein, the term “preventing” in the context of antimicrobial, indicates that the growth rate of the microorganism cells is essentially nullified or is reduced by at least 20%, at least 30%, at least 40%, at least 50%, at least 60%, at least 70%, at least 80%, or at least 90%, including any value therebetween, of the appearance of the microorganism in a comparable situation lacking the presence of the disclosed polymeric material or a composition of matter containing same. Alternatively, preventing means a reduction to at least 15%, 10%, or 5% of the appearance of the microorganism cells in a comparable situation lacking the presence of the disclosed polymeric material or a composition of matter containing same. Methods for determining a level of appearance of a microorganism cells are known in the art.

In some embodiments there is provided an article which comprises the composition of matter incorporated in and/or on a substrate.

Compositions of matter as described herein can be incorporated within any of the articles of manufacturing, during manufacture of any of the article described herein.

The substrates presented herein can be used to modify any industrial or clinical surface to prevent microbial colonization and biofilm formation.

The Process:

The present inventors have designed and successfully practiced processes for preparing the herein disclosed compounds (e.g., MSF), and their corresponding polymer, e.g., Poly(MSF), Poly(ITMS), or any derivative and copolymer thereof as described hereinabove in e.g., Formulae Mc and VI.

It is to note that synthesizing a controlled size of the polymer described hereinthroughout is subjected to various limitations, imposed by a e.g., different tendency of the monomers to disperse in the solution, complicated desired structural features that are required for optimal size, uniformity, and performance of the crosslinked polymer, incompatibility of the reactants, initiators and the like. Hence, devising a process that overcomes these limitations and is designed to obtain uniform cross-linked polymeric microparticles or nanoparticles that exhibits at least a reasonable performance is highly advantageous.

Hence, according to another aspect of embodiments of the invention there is provided a process of synthesizing the composition-of-matter described herein, the process comprising the step of free radical homopolymerization or copolymerization of monomers of one or more of the disclosed the compound in the presence of (a) a free radical initiator; (b) a cross-linking monomer and optionally (c) a stabilizer, thereby making the particle.

In exemplary procedures, the MSF and the diacrylate (TTEGDA) were co-polymerization in dispersion. In some embodiments, the dispersion comprises an aqueous phase. In some embodiments, the dispersion comprises an organic phase. Non-limiting exemplary organic phase is isopropanol.

The polymerization of various monomeric units can be effected by any polymerization method known in the art, e.g., using suitable polymerization initiators and optionally chain transfer agents. Such suitable polymerization initiators and chain transfer agents can be readily identified by a person skilled in the art.

As demonstrated in the Examples section that follows, the polymerization can be performed via a radical polymerization methodology in an aqueous solution.

The term “radical polymerization” or “free radical polymerization” refers to a method of polymerization by which a polymer is formed from the successive addition of free radical building blocks. Free radicals can be formed via a number of different mechanisms usually involving separate initiator molecules. Since the radical polymerization initiator can generate a radical by abstracting hydrogen from a carbon-hydrogen bond, when it is used in combination with an organic material such as a polyolefin a chemical bond can be formed. Following creation of free radical monomeric units, polymer chains grow rapidly with successive addition of building blocks onto free radical sites.

As a radical polymerization initiator for initiated polymerization or redox initiated polymerization, the following exemplary water soluble radical polymerization initiators may be used, without being limited thereto, singly or in a combination of two or more types: peroxides such as ammonium persulfate, potassium persulfate, sodium persulfate, hydrogen peroxide, benzoyl peroxide, cumene hydroperoxide, or di-t-butyl peroxide; a redox initiator that is a combination of the above-mentioned peroxide and a reducing agent such as a sulfite, a bisulfite, thiosulfate, formamidinesulfinic acid, or ascorbic acid; or an azo-based radical polymerization initiator, such as, without limitation, 2,2′-azobis(2-amidinopropane) (AIBN), AIBNCOOH, and 2,2′-azobis(2-amidinopropane), and potassium persulfate (PPS). In exemplary embodiments, the initiator is selected from the group consisting of: PPS and AIBN.

As described hereinabove, it is to be understood that a polymerization process utilizing monomer having a functional group that can form a crosslinked structure.

From the viewpoint of ease of incorporation of the crosslinked structure as described hereinabove under “The Compositions-of-matter”, the method in which a polymerization reaction is carried out using in combination a crosslinking agent (monomer) having at least two polymerizable double carbon-carbon bonds. Similar crosslinking reaction may be caused by heating at the same time as radical polymerization.

It is to be understood that other radical polymerization methodology can be applied, such as, without limitation, living radical polymerization.

By “living polymerization” it is meant to refer to a form of chain growth polymerization where the ability of a growing polymer chain to terminate has been removed. Living radical polymerization is a type of living polymerization where the active polymer chain end is a free radical.

Several methodologies of living radical polymerization are known in the art and are conceivable to be applied in the context of the present invention, including, without limitation, reversible-deactivation polymerization, catalytic chain transfer, cobalt mediated radical polymerization, iniferter polymerization, stable free radical mediated polymerization, atom transfer radical polymerization, reversible addition fragmentation chain transfer (RAFT) polymerization, iodine-transfer polymerization (ITP), selenium-centered radical-mediated polymerization, telluride-mediated polymerization (TERP), and stibine-mediated polymerization.

As described hereinthroughout, in some of any of the embodiments, the polymerization process is affected in an aqueous solution comprising the mixture of the monomers and:

1 to 20%, or 2 to 15%, 1 to 10%, 2 to 5%, 1 to 20%, 1 to 20%, by weight, of the cross-linking monomers;

0.2 to 20%, 0.5 to 20%, 1 to 20%, by weight of a free radical initiator; and 0.1-20%, 0.2 to 20%, 0.5 to 20%, 1 to 20%, by weight of the stabilizer.

As described in the Examples section that follows, the particle size of the crosslinked polymer is affected by the initial weight ratio of monomers in the solution.

In exemplary embodiments, the total concentration (% w/v) of the monomers in the solution is e.g., 1%, 2%, 3%, 4%, 5%, 10%, or 15%, including any value therebetween. As exemplified in the Examples section that follows, the hydrodynamic diameter of the formed polymers may be increased with increasing the total concentration of the monomers.

In exemplary embodiments, the initiator type (e.g., PPS and AIBN) and the concentration thereof may affect the hydrodynamic size and size distribution of the formed crosslinked polymeric nanoparticles.

In exemplary embodiments, the concentration of the initiator, e.g., PPS, or AIBN, is e.g., 1%, 2%, 3%, 4%, 5%, 6%, 7%, 8%, 9%, 10%, including any value therebetween.

In some embodiments, the size and the polymerization yield of the crosslinked polymer is affected by the temperature.

In some embodiments the size and the diameter of the crosslinked polymer is affected by duration (time) of polymerization process.

Further embodiments of parameters affecting the particle size of the polymer are described hereinbelow under the Example section.

In some embodiments, the duration of polymerization process is at least 1 minute. In some embodiments, the duration of polymerization process is at least 30 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, or at least 50 minutes. In exemplary embodiments, the duration of polymerization process is at least 1 minute, e.g., 1 min, 5 minutes or 30 minutes. Each possibility represents a separate embodiment of the invention.

### Definitions

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range; e.g., “21-100”, is stated herein, it implies that the group, in this case the alkyl group, may contain 21 carbon atom, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated.

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)₂—R′ group, where Rx is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a —C≡N group.

As used herein, the term “azide” refers to a —N₃ group.

The term “sulfonamide” refers to a —S(═O)₂—NR′R″ group, with R′ and R″ as defined herein.

The term “phosphonyl” or “phosphonate” describes an —O—P(═O)(OR′)₂ group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

### General

As used herein the term “about” refers to ±10%.

The terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials

The following analytical-grade chemicals were purchased from Sigma-Aldrich (Israel) and used without further purification: thiourea, benzoyl peroxide (BP), chloromethyl styrene (CMS; 97%), divinylbenzene (DVB; 80%), 2-methoxyethanol, ethanol, diethyl ether and polyvinylpyrrolidone (PVP, M_{w} 360,000). G-9/230 Primer from ACTEGA

Coating & Sealants. PP films of A4 size and 30 μm thick were obtained from Dor Film Ltd, Israel. Water was purified by passing deionized water through an Elgastat Spectrum reverse osmosis system (Elga Ltd, High Wycombe, UK).

### Example 1

### Poly(Isothioronium Methylstyrene)

### Synthesis Methods

Synthesis of the Isothioronium Methylstyrene Monomer:

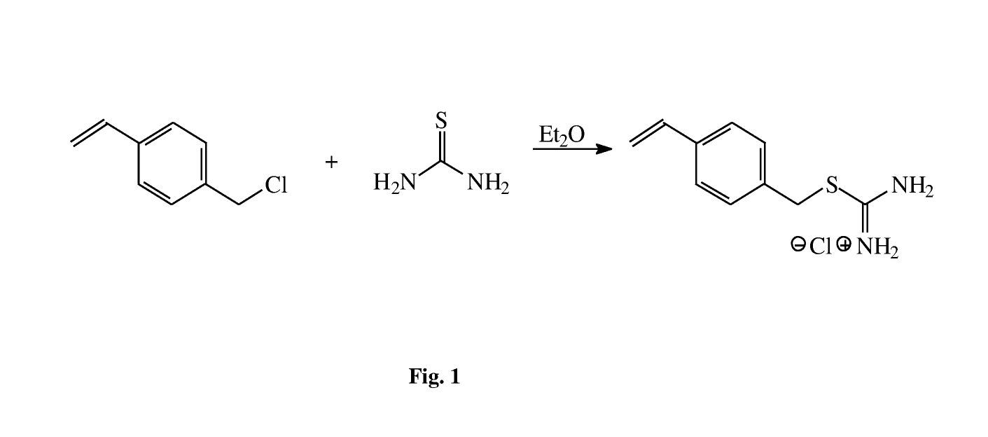

In exemplary procedures, the monomer ITMS was synthesized according to the literature [Nelson S J and Mich G P. S-(4-Vinylbenzyl)-Isothiourea and Isothiouronium Chloride. United States Rubber Company, New York, N.Y., a corporation of New Jersey, 1966. pp. 3, 260, 748.], as shown in FIG. 1.

Briefly, thiourea (0.21 mol) was dissolved in methanol (60 mL), followed by the addition of chloromethylstyrene (CMS, 0.2 mol) to the solution. The reaction mixture was then stirred at room temperature for 24 h. Diethyl ether was then added to precipitate the desired ITMS monomer. The filtered product was purified by dissolving it in ethanol and re-precipitation with ether (41.13 g, 0.18 mol, 90%). The solid residue was analyzed by ¹H and ¹³C NMR which showed the pure desired product.

Nuclear Magnetic Resonance (NMR) spectroscopy was performed on Bruker AC 400 MHz spectrometer. ¹H and ¹³C NMR spectra were recorded in deuterated dimethyl sulfoxide.

1H NMR (400 MHz, DMSO-d6) δH in ppm: 4.53 (s, 2H, CH2-S), 5.28 (d, 10.8 Hz, 1H, CH₂═CH [trans]), 5.85 (d, 17.6 Hz, 1H, CH₂═CH [cis]), 6.73 (dd, 10.8 Hz and 17.6 Hz, 1H, CH₂═CH [gem]), 7.41 (d, 8.2 Hz, Arom-CH), 7.48 (d, 8.2 Hz. Arom-CH), 9.32 (s, 4H, Isothiouronium).

¹³C NMR (400 MHz, DMSO-d₆) δ_{C} in ppm: 34.8 (CH₂—S), 114.9 (CH2═CH), 126.6 and 129.1 (Arom-CH), 133.6 and 135.8 (Arom C), 137.4 (CH₂═CH), 170.3 (C-Isothiouronium).

MS (Cl⁺): 117 (CH₂CHArCH₂⁺, 100%), 193 (M+, 10.5%).

Synthesis of the Polyisothioronium Methylstyrene Microparticles of Narrow Size Distribution:

In a typical experiment, PITMS particles with dry diameter of 376±42 nm were prepared by adding ITMS (0.45 g), DVB (0.05 g), BP (25 mg) and PVP (0.1 g) to 2-methoxyethanol (10 mL). The mixture was shaken at 73° C. for 22 h. The resulting particles were washed for removal of excess reagents by intensive centrifugation cycles with ethanol and then water, and dried by lyophilization. The effect of various polymerization parameters, e.g., monomer, initiator and stabilizer concentrations, on the size and size distribution, and the polymerization yield of the ITMS to produce the particles was also elucidated.

### Characterization Methods

Antibacterial assay: In exemplary procedures, the antibacterial activity of the PITMS microspheres of 376±42 nm diameter was evaluated using the Gram-negative Escherichia coli C600 and Pseudomonas aeruginosa PAO1, and the Gram-positive Staphylococcus aureus FRF1169 and Listeria innocua ATCC 33090, as the experimental models. All the bacterial strains used in this study were grown overnight in Luria Bertani (LB, Difco) media under shaking (250 rpm) at 37° C. On the following day, the overnight cultures were each diluted into twofold concentrated LB medium to obtain a concentration of 2×10⁵ colony-forming units (CFU/mL). The bacterial suspensions were incubated overnight with equivalent volumes of either PITMS particles (2%, 1%, 0.5%) or sterilized water (control). In the following day, 10-fold serial dilutions were carried out and the bacterial cells were plated on LB agar plates, followed by their incubation at 37° C. for 20 h. Cell growth was monitored and determined by viable cell count and expressed as colony forming units (CFU/mL).

Physical and Morphological Characterization of the PITMS Microparticles:

Fourier Transform Infrared (FTIR) analysis was performed with a Bruker Platinum-FTIR QuickSnap™ sampling modules A220/D-01. The analysis was performed with 13 mm KBr pellets that contained 2 mg of the detected material (ITMS or PITMS and 198 mg KBr. The pellets were scanned over 50 scans at a 4 cm⁻¹ resolution.

Electrokinetic properties zeta-potential (ζ-potential) as a function of pH were determined with Zetasizer (Zetasizer 3000HSa, Malvern Instruments, UK). ζ-potential measurements were performed at a constant ionic strength of 0.1 M.

Dried particle size and size distribution were measured with a Scanning Electron Microscope (SEM). SEM pictures were obtained with a JEOL, JSM-840 Model, Japan. For this purpose, a drop of dilute particles dispersion in distilled water was spread on a glass surface, and then dried at room temperature. The dried sample was coated with carbon in vacuum before viewing under SEM. The average particle size and distribution were determined by the measurement of the diameter of more than 200 particles with image analysis software (Analysis Auto, Soft Imaging System GmbH, Germany).

Hydrodynamic diameter and size distribution of the particles dispersed in double distilled (DD) water were measured at room temperature with a particle analyzer; model NANOPHOX (SympatecGmbH, Germany).

The weight % polymerization yield of the ITMS to form PITMS microparticles was calculated by the following expression:

Polymerization yield (weight %)=[W(PITMS)/W(PITMS+DVB)]×100

where W(PITMS) is the weight of the dried PITMS particles and W(ITMS+DVB) is the initial weight of the ITMS and DVB monomers.

### Results

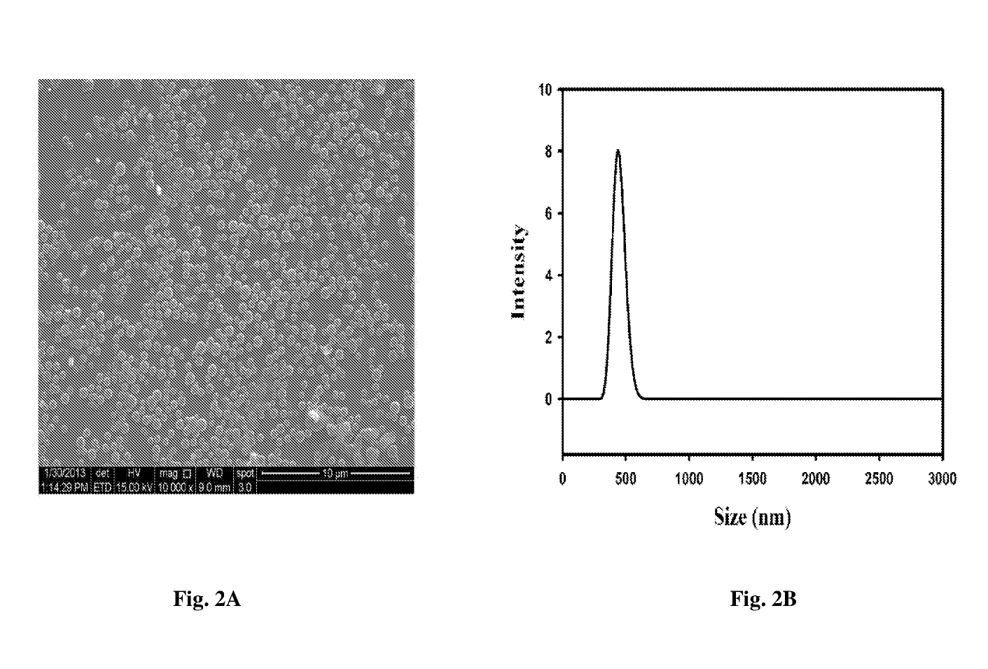

Crosslinked PITMS microparticles of narrow size distribution were prepared by dispersion co-polymerization of ITMS and DVB according to the description in the experimental part. The polymerization yield of the obtained PITMS microparticles was calculated to be 85%. FIG. 2 presents a SEM image (FIG. 2A) and a typical hydrodynamic size histogram (FIG. 2B) of the obtained PITMS microparticles. The dry diameter and size distribution of these PITMS particles, as shown by the SEM image, are 376±42 nm, while the hydrodynamic diameter and size distribution of these particles dispersed in 2-methoxyethanol, as shown by the size histogram, are 443±55 nm. The hydrodynamic diameter is slightly larger than the dry diameter since it also takes into account Brownian motion, absorbed solvent or water, and surface-adsorbed solvent or water molecules.

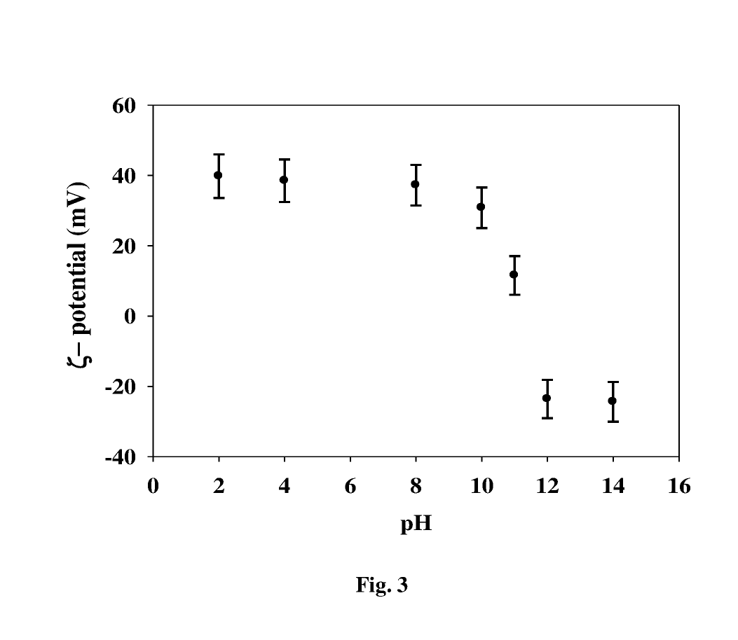

Without being bound by any particular theory, the ζ-potential of the particles may affect their stability, that is, a positive particle surface charge will create repulsion between the particles and may prevent aggregation. FIG. 3 illustrates a slight decrease in the ζ-potential of the particles from 40 mV to 32 mV by increasing the pH of the aqueous continuous phase from 2 to 10. A further increase in the pH of the continuous phase, e.g., from 10 to 12, leads to a sharp decrease in the ζ-potential of the particles from 32 mV to −23.6 mV. In an acidic environment, the particle surface possesses positively charged isothioronium groups, and as the pH increases, the surface charge decreases as illustrated in FIG. 3. At the isoelectric point (around pH 11 as shown in FIG. 3), the particles are unstable due to aggregation.

Without being bound by any particular theory, it is assumed that increasing the pH of the continuous phase above 11.5 is likely to cause hydrolysis of the isothioronium groups involving deprotonation of thiol groups.

### Example 2

### Effect of Polymerization Parameters on the Size and Size Distribution of the PITMS Microparticles

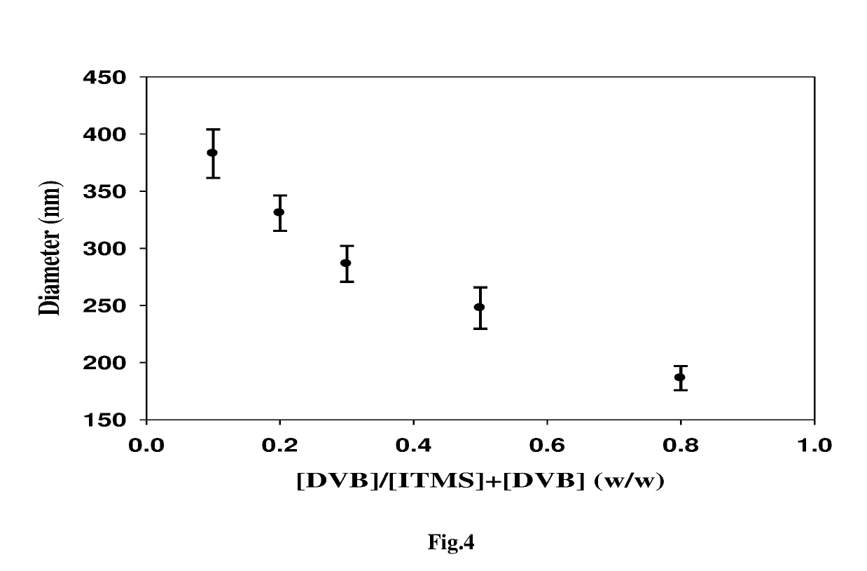

Effect of the DVB concentration: In exemplary procedures, the effect of the weight ratio [DVB]/[ITMS+DVB] on the hydrodynamic diameter and size distribution of the formed PITMS microparticles was studied while retaining a constant total monomer ([ITMS]+[DVB]) concentration (0.5 g). Increasing the weight ratio of [DVB]/[ITMS]+[DVB] results in a decrease in diameter and size distribution of the formed PITMS particles (as shown in FIG. 4). For example, raising the ratio from 0.1 to 0.3 and 0.8 decreases the particle size and size distribution from 383±42 to 286±30 and 186±20 nm, respectively.

Without being bound by any particular theory, it is assumed that this behavior may be explained the fact that increasing the crosslinker concentration decreases the ability of the growing nuclei to swell, resulting in smaller particles.

Effect of Initiator Concentration:

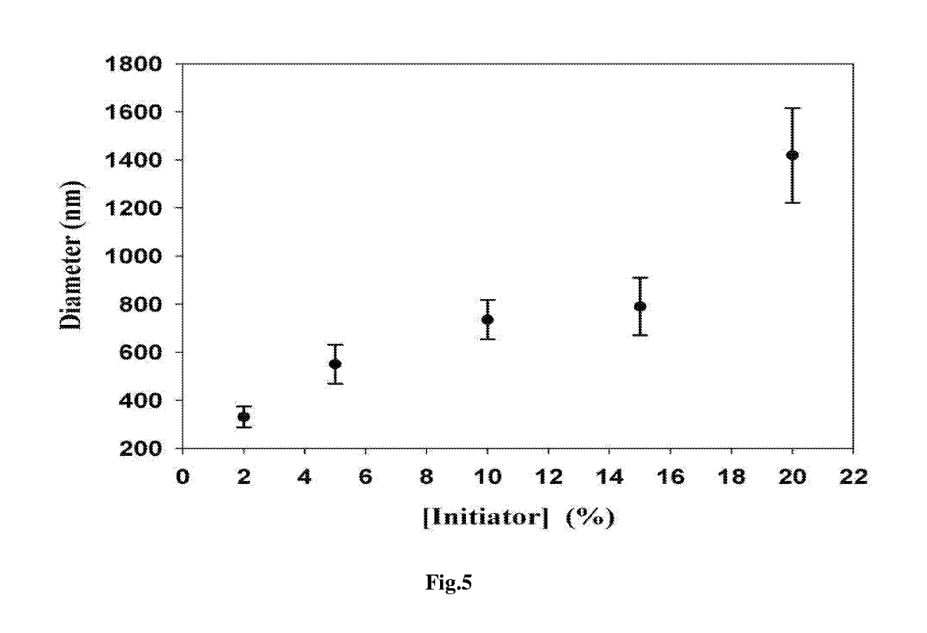

The effect of the BP concentration on the hydrodynamic diameter and size distribution of the PITMS particles was also elucidated. Increasing the BP concentration resulted in an increase in the diameter and size distribution of the formed PITMS particles. For example, raising the weight % of BP concentration from 2 to 10 and 15% increased the particle size and size distribution from 331±44 to 735±82 and 790±119 nm, respectively (as shown in FIG. 5).

Without being bound by any particular theory, it is assumed that increasing the initiator concentration causes an increase in the oligomer radical concentration, and thus, in the concentration of precipitated oligomer chain. Because of this and the slow adsorption of the stabilizer, the aggregation process is enhanced, resulting in larger particles. The increase in the size distribution as the initiator concentration increases may be explained by the increase in the number of the oligomeric chains as the initiator concentration increases, thus favoring secondary nucleation during the particle growth stage, which increases the particle size distribution.

Effect of Stabilizer Concentration:

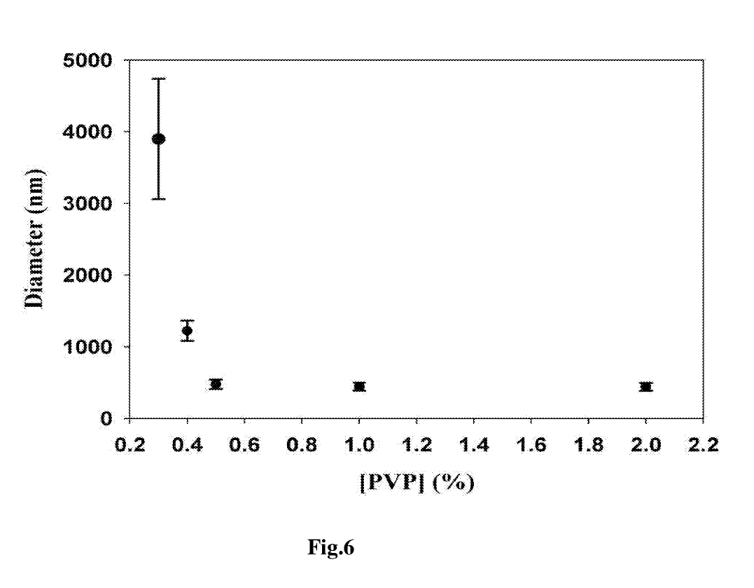

The effect of the PVP concentration on the hydrodynamic diameter and size distribution of the PITMS particles was determined, and it was shown that increasing the stabilizer concentration leads to the formation of smaller PITMS particles with narrower size distributions as shown in FIG. 6. For example, an increase in the PVP concentration from 0.3 to 0.5 and 2% leaded to a decrease in the diameter and size distribution of the PITMS particles from 3898±840 to 490±60 and 441±54 nm, respectively.

Without being bound by any particular theory, the inverse behavior of the stabilizer concentration and the size and size distribution of the formed particles can be explained by the fact that increasing the stabilizer concentration increases the adsorption of the stabilizer to the surface of the nuclei leading to greater protection against the growing process and thus leading to smaller particle sizes and narrower size distributions.

Antibacterial Activity of the PITMS Microparticles:

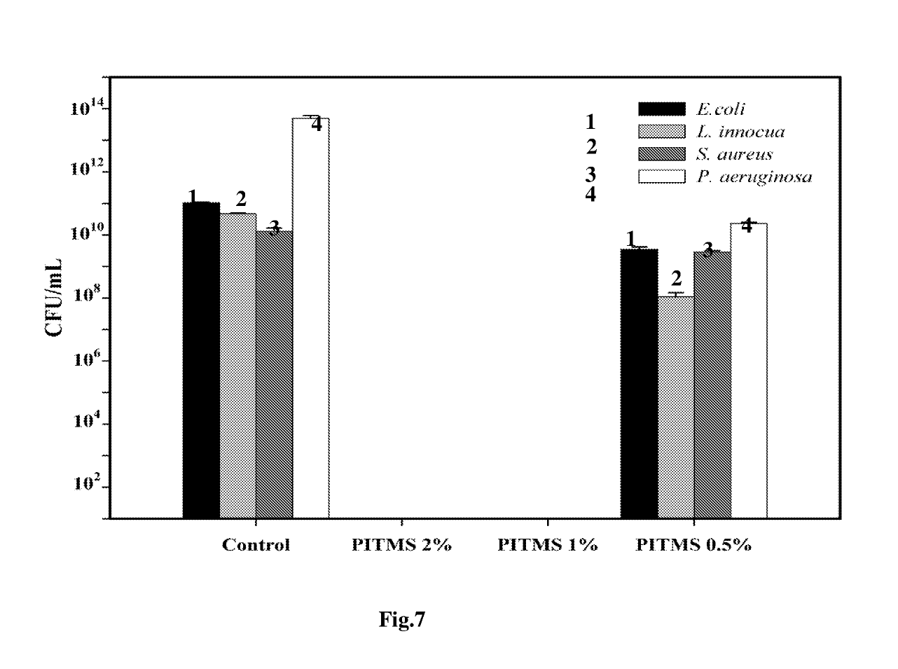

In exemplary procedures, the antibacterial properties of the crosslinked PITMS microspheres of 376±42 nm were tested against E. coli, P. aeruginosa, S. aureus, and L. innocua, four common bacterial pathogens. As shown in FIG. 7, PITMS particles at concentrations of both 1% and 2%, incubated for 24 h with each of the four bacterial strains, resulted in killing of all the bacteria, compared to the negative control, consisting of bacterial suspensions in LB medium that were exposed to double distilled water. This suggested potent bactericidal activity of the PITMS particles. However, at a concentration of 0.5% only partial bactericidal activity was observed, implying that 1% is the PITMS MBC needed for achieving total killing under these experimental conditions.

### Example 3

### Poly(Isothioronium Methylstyrene) NPs

### Preparation Method

The monomer ITMS was synthesized as described hereinabove.

In a typical experiment, PITMS NPs with dry diameter of 67±8 nm were prepared by adding ITMS (425 mg), EGDMA (75 mg), PPS (25 mg) and Tween 20 (100 mg) to water (10 mL). The mixture was shaken at 73° C. for 15 h. The obtained NPs dispersed in water were then isolated from impurities by dialysis. The effect of various polymerization parameters, e.g., total monomers, initiator and crosslinker concentrations, on the size and size distribution, and the polymerization yield of the ITMS to produce the particles was also elucidated.

Coating of the PET Films with the PITMS NPs:

PITMS NPs of 67±8 nm diameter dispersed in water (4%) were first dispersed in the G-9/230 film former 4% aqueous solution (1:1 v/v). The obtained aqueous dispersion was then spread on the 23 μm thick PET films with a Mayer rod hand coater, followed by drying the PITMS coating on the PET films over night at room temperature.

### Characterization Methods

Antibacterial assay: The antibacterial activity of the PITMS NPs of 67±8 nm diameter was evaluated using the Gram-negative bacteria E. coli C600 and P. aeruginosa PAO1, and the Gram-positive bacteria S. aureus FRF1169 and L. innocua ATCC 33090, as the experimental models, according to the procedures described hereinabove.

Static Biofilm Formation Assay:

The antibiofilm activity of the PET/PITMS films against Listeria bacteria was evaluated and compared to a control PET film and a PET film coated with the film former only using the Gram-positive bacteria Listeria ATCC 33090 as the experimental model. Bacteria were grown overnight in Tryptic Soy Broth (TSB, DIFCO) growth medium. On the following day, bacterial cells were diluted in TSB to obtain a working solution with an OD₅₉₅ of 0.3 (approximately corresponds to 3*10⁸ CFU/ml). 1 ml from the stock solution was taken into each well in a 24-well plate (DE-GROOT). Each of the different films was added to the well (1 cm diameter). The plates were then incubated at 25° C. under gentle agitation (100 rpm) for 20 h. In the day after, the films were rinsed 3 times with distilled water to remove the unattached bacteria (i.e. planktonic cells) and subsequently the attached cells were scraped from the films using 250 μl of Tris-HCl (0.1M, pH 7.2) and cell scrapers (Greiner Bio-one). 200 μl out of the 250 μl, used for scrapping the cells, were transferred into the first line of a 96-well plate (Greiner Bio-One), while the rest of the lines were filled with 180 μl of Tris-HCl (0.1M, pH 7.2). Serial dilutions were carried out and the cells spotted onto NB agar plates, which were then incubated at 37° C. for 20 h. Cell growth was monitored and determined by a viable cell count. The experiments were conducted at least three independent times, with internal duplicates.

Characterization of the PITMS NPs and the PET/PITMS Films:

Fourier Transform Infrared (FTIR) analysis was performed with a Bruker Platinum-FTIR QuickSnap™ sampling modules A220/D-01. The analysis was performed with 13 mm KBr pellets that contained 2 mg of the detected material (ITMS or PITMS and 198 mg KBr. The pellets were scanned over 50 scans at a 4 cm⁻¹ resolution.

Electrokinetic properties (*-potential) as a function of pH were determined with Zetasizer (Zetasizer 3000HSa, Malvern Instruments, UK). ζ-potential measurements were performed at a constant ionic strength of 0.1 M.

Dried particle size and size distribution were measured with a Transmission Electron Microscope (TEM). SEM pictures were obtained with a JEOL, JSM-840 Model, Japan. For this purpose, a drop of dilute particles dispersion in distilled water was spread on a glass surface, and then dried at room temperature. The dried sample was coated with carbon in vacuum before viewing under SEM. The average particle size and distribution were determined by the measurement of the diameter of more than 100 particles with image analysis software (Analysis Auto, Soft Imaging System GmbH, Germany).

Hydrodynamic diameter and size distribution of the particles dispersed in double distilled (DD) water were measured at room temperature with a particle analyzer; model NANOPHOX (SympatecGmbH, Germany).

The thermal behavior of the PITMS NPs was determined by thermo gravimetric analysis (TGA) with a TA TGA Q500 instrument combined with mass spectrometer (MS) from Thermo-star Pfeiffer Inc.

The weight % polymerization yield of the ITMS to form PITMS NPs was calculated by the following expression:

Polymerization yield (weight %)=[W(PITMS)/W(ITMS+EGDMA)]×100

where W(PITMS) is the weight of the dried PITMS NPs and W(ITMS+EGDMA) is the initial weight of the ITMS and EGDMA monomers.

Film Thicknesses Measured on a Millitron 1204 IC (Mahr Feinmesstechnik GmbH):

The optical parameters transmittance, haze, and clarity of the films were measured on a BYK Gardner haze-gard plus in accordance with ASTM D1003 “Standard Test Method for Haze and Luminous Transmittance of Transparent Plastics”. The PET, PET/film former and PET/PITMS films were irradiated with visible light; the transmitted intensity was then integrated by the instrument.

Haze and clarity are per definition components of scattered light under wide angle) (>2.5° and narrow angle (<2.5°), respectively. Mean values and standard deviations of transmittance, haze, and clarity were obtained by taking the average over several measurements (at least 4 measurements each).

Migration Test:

A specimen of 0.5 dm² of each film (PET, PET/film former and PET/PITMS films) was incubated with 50 mL of 3% acetic acid in distilled water or 95% ethanol for 2 h at 70° C. The migration of the NPs from the PET/PITMS films into the continuous phase was accomplished by weighing the PET/PITMS films before and after the incubation and measuring the absorbance spectrum of the filtrate. In addition, the haze of the PET/PITMS films during time was also measured.

### Results

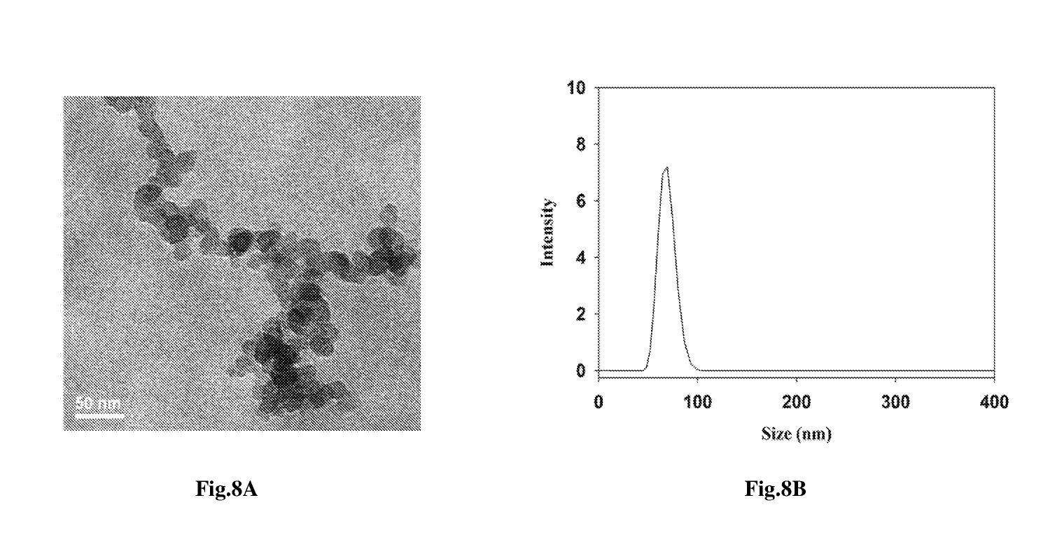

PITMS NPs of narrow size distribution were prepared by dispersion co-polymerization of ITMS and EGDMA according to the experimental part. The formed PITMS particles were washed with water at 60° C. in order to remove traces of the monomers and excess reagents. The polymerization yield of the obtained PITMS NPs was calculated to be 75%. FIGS. 8A-B present a TEM image (FIG. 8A) and a typical hydrodynamic size histogram (FIG. 8B) of the obtained PITMS NPs. The dry diameter and size distribution of these PITMS particles, as shown by the TEM image, are 19±2 nm, while the hydrodynamic diameter and size distribution of these particles dispersed in water, as shown by the size histogram, are 67±8 nm. The hydrodynamic diameter is larger than the city diameter probably since it also takes into account swollen and surface-adsorbed water molecules.

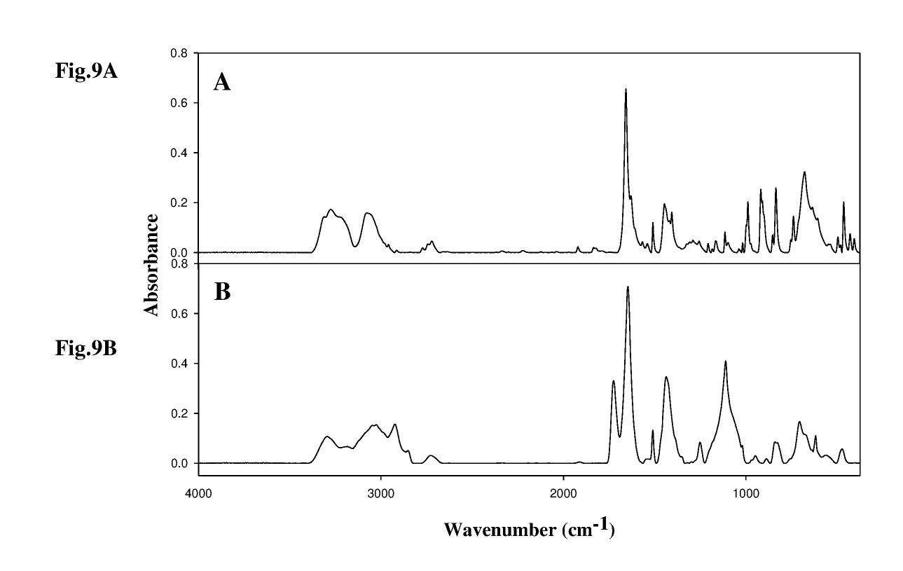

FTIR spectra of the ITMS monomer (A) and the PITMS NPs (B) are shown in FIGS. 9A-B, respectively. The FTIR spectrum of the PITMS NPs is similar to that of the monomer, except for the absorption peak at about 916 and 987 cm⁻¹ corresponding to the vinylic C—H bending band indicating the lack of residual monomer within the polymeric particles. Instead, the peaks that appears at 1110 and 1730 cm⁻¹ corresponding to the C—O and C═O stretching band of EGDMA.

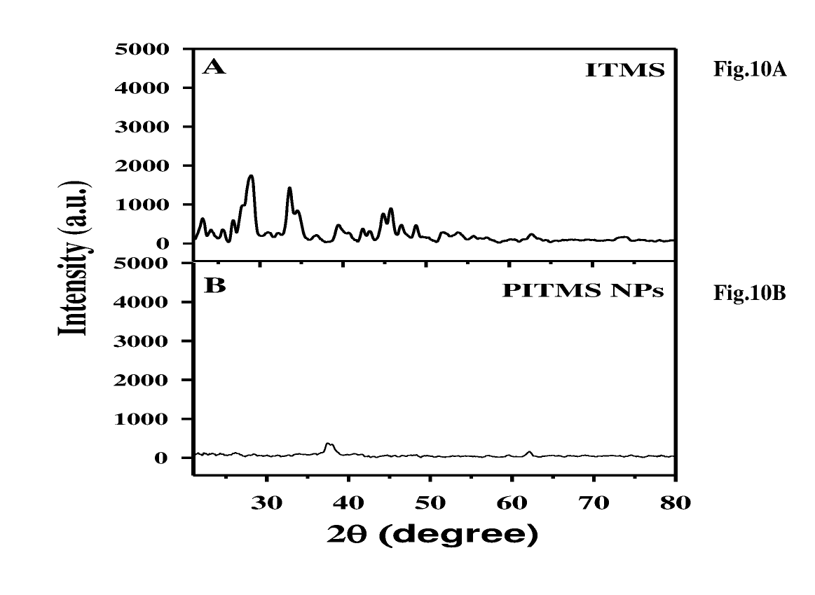

X-ray diffraction patterns of the ITMS monomer and the PITMS NPs are illustrated in FIG. 10. Clear, sharp, and narrow diffraction peaks typical of crystalline materials are displayed in FIG. 10A. These X-ray powder diffraction patterns indicate the crystalline nature of the monomer. In contrast, the X-ray powder diffraction pattern of the PITMS NPs (FIG. 10B), suggests the existence of a fully amorphous phase of the polymer, probably due to the loss of the crystalline structure of the monomer by the radical polymerization process.

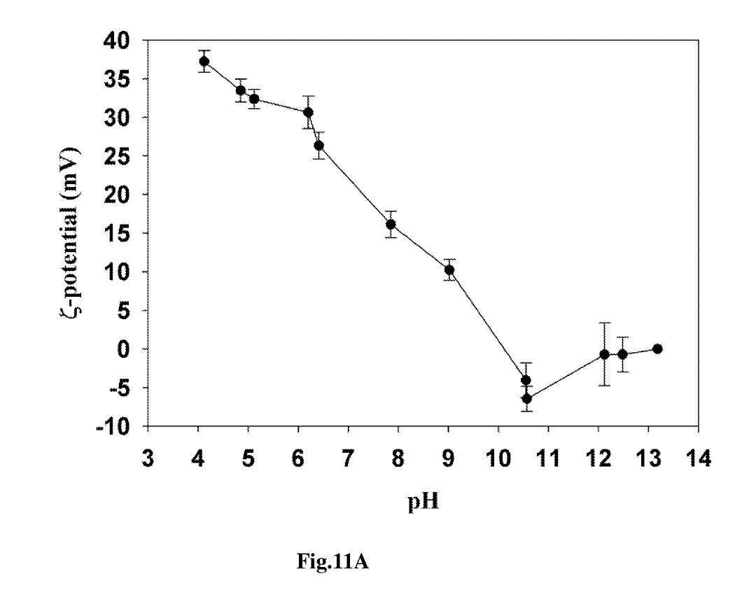

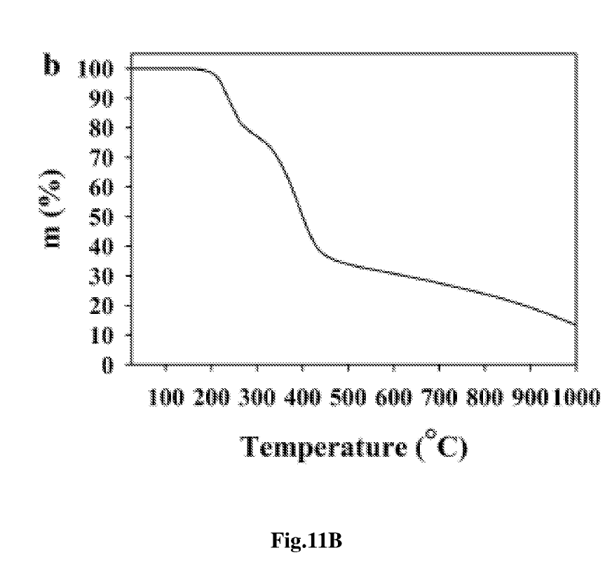

As described above, the ζ-potential of the particles may affect their stability, that is, a positive particle surface charge will create repulsion between the particles and may prevent aggregation. FIG. 11A illustrates a consistent sharp decrease in the ζ-potential of the nanoparticles by increasing the pH of the aqueous continuous phase from 37 mV at pH 4.0 to −6.0 mV at pH 10.5. At the isoelectric point (around pH 10.2 as shown in FIG. 11A), the particles are not stable due to an aggregation process. Increasing the pH of the continuous phase above 11.5 causes probably, and without being bound by a particular theory, to hydrolysis of the isothioronium groups to deprotonated thiol groups.

The thermal stability is an important factor when incorporating an external substance as an additive to polymer matrices. The thermal stability of the PITMS NPs aqueous dispersion, after drying, was evaluated by TGA, as illustrated in FIG. 11B. No mass loss was observes between room temperature to 160° C. indicating the thermal stability of the NPs in this temperature range. In the range of 160-270° C. and 270-450° C., mass loss of 22% and 41% was observed, attributed to the degradation of thiourea hydrochloride and the aromatic group from the PITMS NPs, respectively, as was indicated by the MS. In the range of 450-1000° C., mass loss of 22% was observed, probably attributed to the degradation of the polymer crosslinked carbon chain.

### Example 4

### Effect of Polymerization Parameters on the Size and Size Distribution of the PITMS NPs

Effect of the EGDMA Concentration:

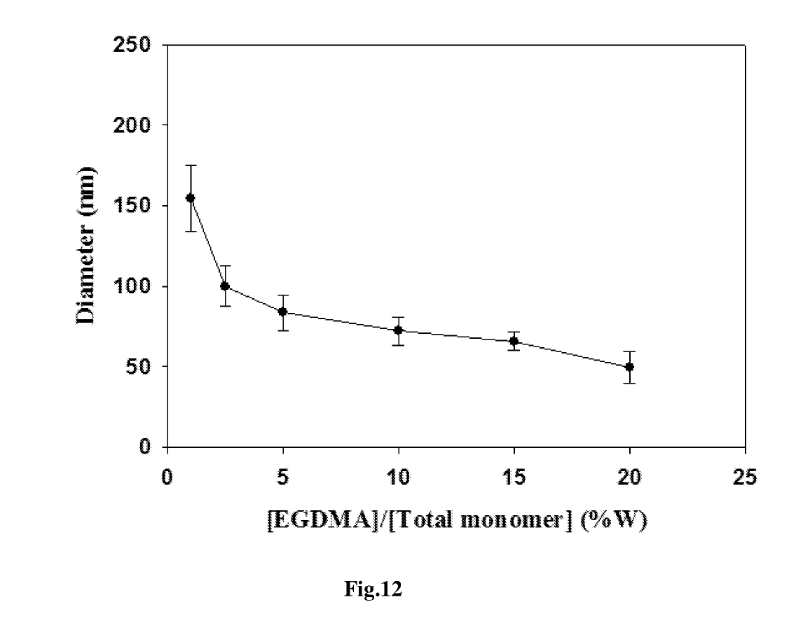

The effect of the weight ratio [EGDMA]/[ITMS+EGDMA] on the hydrodynamic diameter and size distribution of the formed PITMS NPs was studied while retaining a constant total monomer ([ITMS]+[EGDMA]) concentration (0.5 g). FIG. 12 shows that as the weight ratio of [EGDMA]/[total monomers] increases the diameter and the size distribution of the formed PITMS particles decreases. For example, raising the ratio from 1 to 2.5 and 5% leads to a decrease in the average particle size from 155±21 to 100±13 and 83±11 nm, respectively.

Effect of the Initiator Concentration:

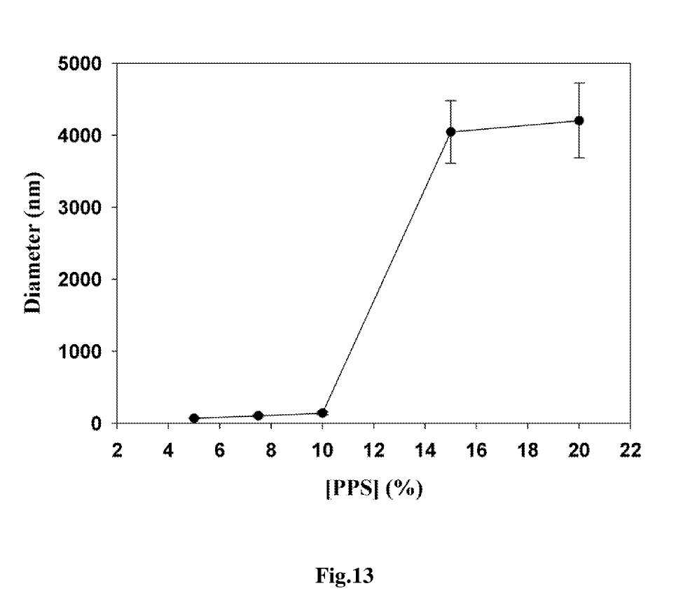

The effect of the PPS concentration on the hydrodynamic diameter and size distribution of the PITMS particles was also elucidated. Increasing the PPS concentration leads to an increase in the diameter and size distribution of the formed PITMS particles as shown in FIG. 13. For example, raising the weight % of PPS concentration from 5 to 7.5 and 10% leads to an increase in the average particle size and size distribution from 70±8 to 100±12 and 139±19 nm, respectively (FIG. 13).

Effect of the Total Monomer Concentration:

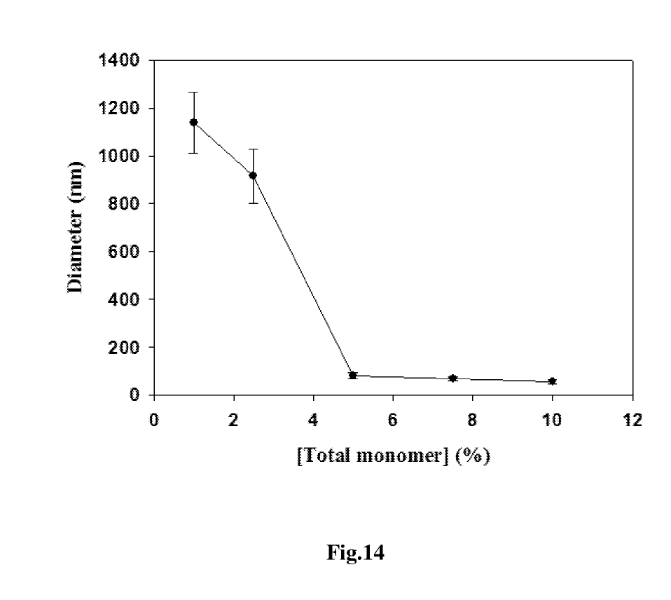

The effect of total monomers concentration ([ITMS]+[EGDMA]) on the hydrodynamic diameter and size distribution of the PITMS particles showed that increasing the total monomer concentration leads to the formation of smaller PITMS particles with narrower size distributions as shown in FIG. 14. For example, an increase in the total monomer concentration from 2.5 to 5 and 7.5% leads to a decrease in the diameter and size distribution of the PITMS particles from 916±113 to 81±12 and 67±9 nm, respectively.

It is assumed, without being bound by a particular theory that increasing the monomer concentration can affect the initial solvency of the reaction medium by decreasing or increasing (depending on the monomer type and the continuous phase) the solubility of the forming oligomers, so they can have shorter or longer chain lengths before precipitating. Earlier precipitation of the shorter oligomers eventually results in a larger number of smaller particles. In addition, increasing the monomer concentration can decrease or increase the solubility of the stabilizer, thus increasing or decreasing its adsorption on the growing particle. Both effects may contribute to the decrease or increase in the particle size.

### Characterization Methods

Antibacterial Activity of the PITMS NPs:

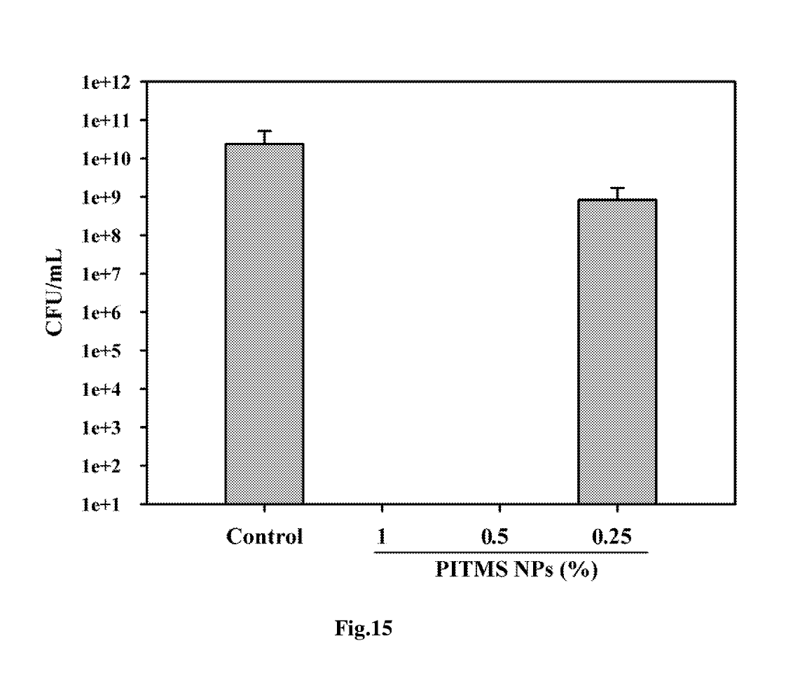

The antibacterial properties of the PITMS NPs of 67±8 nm were tested against Listeria bacteria, a common food born pathogen. As shown in FIG. 15, both 1 and 0.5% PITMS NPs were able to kill all the tested bacteria following 24 h exposure, as opposed to the water-treated control, suggesting a potent antimicrobial activity of the PITMS particles. A 0.25% particle concentration had only a partial bactericidal effect, implying that 0.5% is the minimum inhibitory concentration (MIC) for PITMS needed to inhibit growth under these experimental conditions. Similar results were obtained for three additional pathogenic bacteria: E. coli, P. aeruginosa and S. aureus as shown in the supporting information.

Antibiofilm Activity of the PITMS NPs:

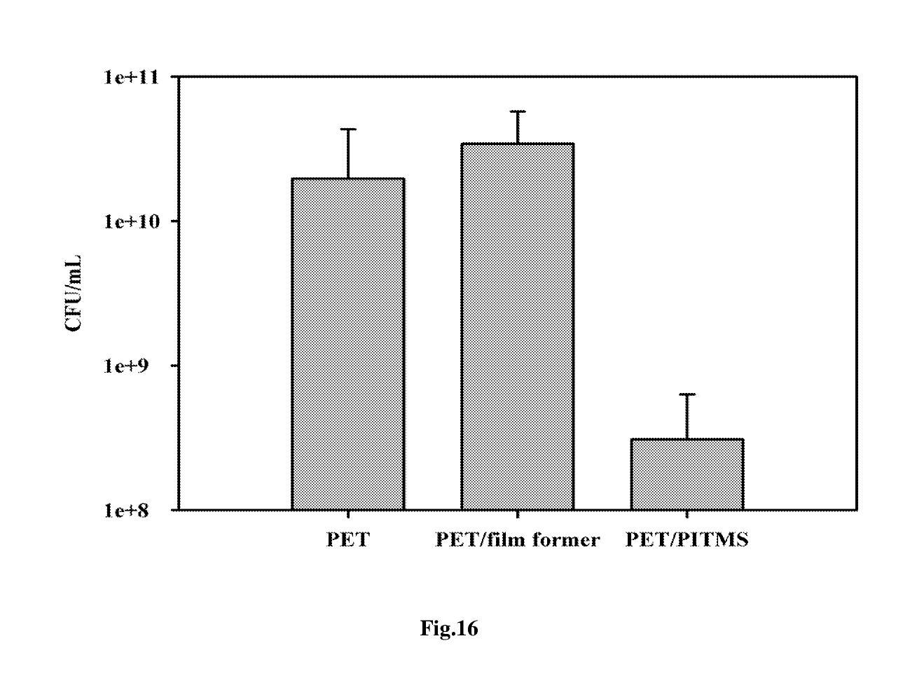

In light of the antibacterial activity exerted by the PITMS, it was sought to determine whether these NPs can also inhibit the biofilm formation of Listeria. Hence, PET films was coated with the NPs aqueous dispersion using a formulation with a ratio of 1:1 PITMS polymer to the film former (G-9/230). A knife (Mayer rod) of 6 μm (wet thickness) was used for the coating. As shown in FIG. 16, a significant reduction in the biofilm formation of Listeria was detected; 2 logs for the PET/PITMS films, in comparison to a film containing the film former only or to a non-coated PET film.

Migration Experiments:

Migration refers to the escape of additives from a polymeric host which may limit the use of additives in plastic especially for food packing applications, pharmaceutical and other hygienic products. Crosslinked NPs that are compatible with the PET film and the film former may overcome this disadvantage, due to their large spatial structure, which reduces their migration while maintaining the activity. Hence, using appropriate NPs as an additive to films will result in antibacterial properties and with decreased extractability and volatility.

Indeed, no migration of the PITMS NPs to the continuous phase composed of 3% aqueous acetic acid or 95% ethanol were found.

The optical properties of polymeric films are important for example in transparent food packaging. The haze, clarity and transmittance of the PET/PITMS in comparison to a film containing the film former only or to a non-coated PET film are shown in Table 1. The results indicate the potential use of the PET/PITMS films as transparent film. There was no change in the optical properties of the various films after a year indicating that there is no migration during this time period.

TABLE 1

| | | | Transmission |
|---|---|---|---|
| | Haze (%) | Clarity (%) | (%) |
| PET | 1.3 ± 0.1 | 99.2 ± 0.0 | 89.5 ± 0.1 |
| PET/film former | 2.5 ± 0.2 | 98.9 ± 0.04 | 90.6 ± 0.5 |
| PET/PITMS | 6.5 ± 0.4 | 94.5 ± 0.7 | 91.0 ± 0.1 |

### Example 5

### Cytotoxicity of the PITMS NP Coating

Cytotoxicity of the PITMS NP Coating:

In exemplary procedures, in vitro cytotoxicity of the PITMS NP coatings was tested by using HaCaT cell line. HaCaT cell line is a spontaneously transformed human epithelial cell line from adult skin and the first permanent epithelial cell line that exhibits normal differentiation.

The cell line is adherent to the used culture dishes. HaCaT cells were grown in DMEM-eagle that was supplemented with 10% heat-inactivated fetal bovine serum (FBS), 1% glutamine and 1% penicillin/streptomycin. Cytotoxicity was performed in two steps. First, the PET, PET/film former and PET/PITMS films were incubated within the medium at 37° C. for 24 h in a humidified 5% CO₂ incubator. The next step is incubation of the supernatant with HaCaT cell line at 37° C. for 48 h and then measuring the release of cytoplasmic lactate dehydrogenase (LDH) into the cell culture supernatants. LDH is an intracellular enzyme that catalyzes the reversible oxidation of lactate to pyruvate. Since LDH is mainly present in the cytosol, it is released into the supernatant only upon cell damage or lysis.

In additional exemplary procedures, cell cytotoxicity was assessed by measuring the release of LDH into cell culture supernatants. LDH activity was assayed using the Cytotoxicity Detection Kit according to the manufacturer's instructions. Cells (3×10⁵ cells per well) were seeded and grown to 75-80% confluency in 96 well plates before treatment with the films supernatants. Cell cultures that were not exposed to the films supernatants were included in all assays as negative controls. Cell cultures that were treated with 1% Triton-x-100 were used as positive controls. The cell cultures were further incubated at 37° C. in a humidified 5% CO₂ incubator and then checked for cellular cytotoxicity after of 48 h. The percentage of cell cytotoxicity was calculated using the formula shown in the manufacturer's protocol. All samples were tested in tetraplicates.

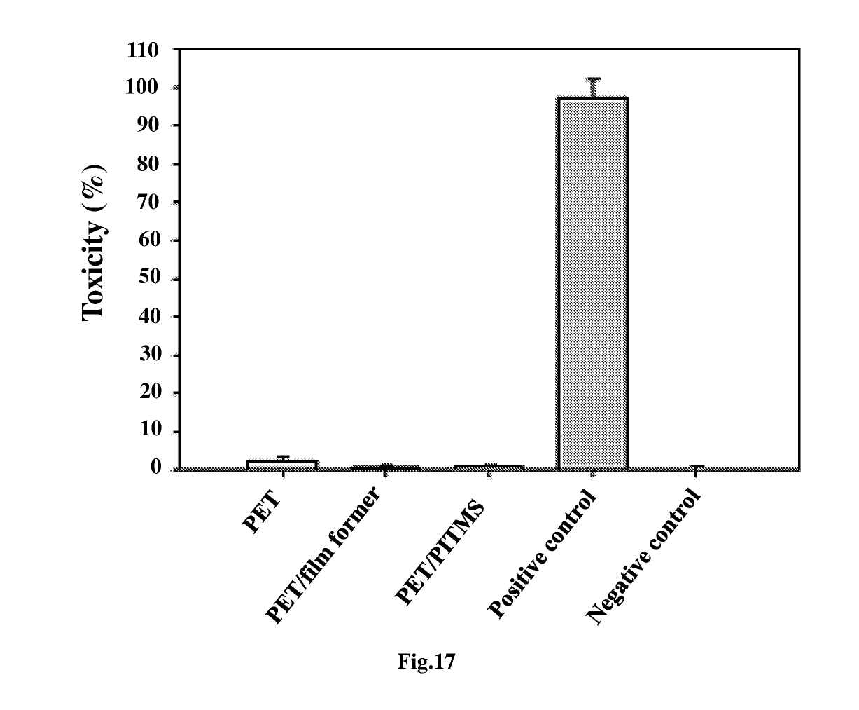

The results showed that when tested by the LDH quantitative assay, all the pre-incubated PET films had no cytotoxic effect on the HaCaT cell line (FIG. 17). PET/PITMS films are therefore suitable for food application, considering their non-toxicity.

### Example 6

### Synthesis and Characterization of the Methyl Styrene Farmin (MSF) Monomer

### Materials

The following analytical-grade chemicals were purchased from commercial sources and used without further purification: 4-vinyl benzyl chloride (CMS), tetra(ethylene glycol) diacrylate (TTEGDA), polyvinylpyrrolidone (PVP, m.w. 360 kDa), sodium hydroxide (NaOH, 1N) from Sigma (Rehovot, Israel); sodium carbonate from Bio-Lab (Jerusalem, Israel); farmin DM4250 from Kao Chemicals (Philippines); 4,4′-azobis(4-cyanovaleric acid) (AIBN—COOH) from Tzamal D-Chem Laboratories Ltd. (Petah Tikva, Israel); dichloromethane from Romical Ltd. (Beer-Sheva, Israel). Water was purified by passing deionized water through an Elgastat Spectrum reverse osmosis system (Elga, High Wycombe, UK).

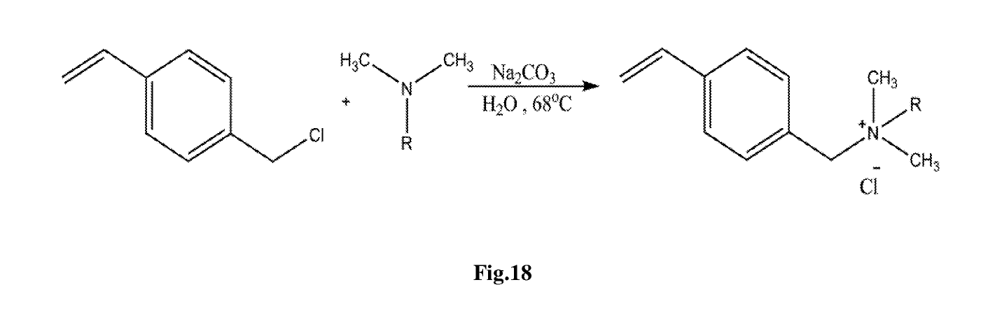

The MSF monomer was synthesized as described in FIG. 18. Sodium carbonate (35 mg, 0.33 mmol) was dissolved in distilled water (5.5 mL). Farmin (5.2 mL, 17 mmol) was then added under a nitrogen atmosphere and the mixture was heated to 68° C. for 1 h. Then, 4-vinyl benzyl chloride (CMS) (3 mL, 20 mmol) was added and the mixture was stirred until a clear solution was obtained. Phase separation was performed by dichloromethane and the desired MSF monomer was precipitated by evaporation of the solvent (92%). The product was analyzed by ¹H NMR, TOF MS⁺ and FTIR, confirm the formation of the desired product.

¹H NMR (400 MHz, MeOD, δ, ppm): 7.75-7.63 (m, 4H), 6.93 (dd, J=8.8 Hz, 1H), 6.04 (d, J=8.8 Hz, 1H), 5.50 (dd, J=5.4 Hz, 1H), 4.66 (s, 2H), 3.16 (s, 6H), 2.01 (s, 2H), 1.42 (d, 22H), 1.027 (t, 3H).

FTIR (KBr pellets, cm⁻¹): 3000 (aromatic C—H), 2850 and 2920 (aliphatic C—H), 1630 (C═C), 1375 and 1465 (sp³C—H).

TOF MS⁺ (m/z): 386 (R═C16, 96%), 358 (R═C14, 100%), 330 (R═C12, 23%).

### Example 7

### Synthesis and Characterization of the Poly(Methyl Styrene Farmin) NPs

### Methods

Synthesis:

In a typical experiment, PMSF NPs with a dry diameter of 40±9 nm were prepared by dispersion co-polymerization of MSF and TTEGDA. For this purpose, MSF (237.5 mg), TTEGDA (12.5 mg), AIBN—COOH (12.5 mg) and PVP (25 mg) were dissolved in water (5 mL). The mixture was purged with N₂ to exclude air and then shaken at 80° C. for 24 h. Extensive dialysis cycles with water was used to wash the resulting NPs of excess reagents. NPs with a dry diameter of 58±29 nm were prepared similarly in absence of PVP. The effect of various polymerization parameters on the size and size distribution of the PMSF NPs was also studied. The polymerization yield of the PMSF NPs was calculated by the following equation:

Polymerization yield (% weight)=W(PMSF)/W(MSF+TTEGDA)×100

where W(PMSF) is the weight of the PMSF dried particles and W(MSF+TTEGDA) is the initial weight of the MSF and TTEGDA monomers.

Bacterial Cultures and Growth Conditions

Escherichia coli ATCC 8739, Listeria innocua ATCC 33090, Staphylococcus aureus FRF1169 and Pseudomonas aeruginosa PAO1 were grown overnight at 37° C. under agitation (250 rpm) in Nutrient Broth (NB, Sigma) and Brain Heart (Difco) growth mediums, respectively.

Antibacterial Activity Assay of the PMSF NPs:

The antibacterial activity of the PMSF NPs was evaluated by determining the minimum inhibitory concentration (MIC) values for all the bacterial strains tested. The stock solution of the PMSF NPs was diluted in two-fold serial dilutions ranging from a concentration of 10 mg/mL to 0.08 mg/mL in NB medium in a 96-well plate (Greiner Bio-one). Each well contained 10⁵ colony-forming units (CFU)/mL of each bacteria, and bacteria treated with double-distilled water (DDW), served as a negative control. The bacterial growth was monitored via absorbance measurements at OD₅₉₅ taken with a microplate reader (Synergy 2, BioTek instruments). All experiments were conducted in duplicates at least three independent times.

### Physical and Morphological Characterization

Nuclear Magnetic Resonance (NMR) spectroscopy was performed on a Bruker AC 400 MHz spectrometer. ¹H NMR spectra were recorded in methanol-D solutions.

Fourier Transform Infrared (FTIR) analysis was performed with a Bruker Platinum-FTIR Quick-Snap™ sampling modules A220/D-01. The analysis was performed with 13 mm KBr pellets that contained 2 mg of the detected material (MSF or PMSF) and 198 mg KBr. The pellets were scanned over 48 scans at a 4 cm⁻¹ resolution.

Dried particle size and size distribution were measured with a Transmission Electron Microscope (TEM). TEM images were obtained with a FEI TECNAI C2 BIOTWIN electron microscope with 120 kV accelerating voltage. For this purpose, a drop of dilute particles dispersion in distilled water was placed on a 400-mesh carbon-coated copper grid, and then dried at room temperature. The average particle size and size distribution were determined by the measurement of the diameter of more than 200 particles with image analysis software (Image J).

Hydrodynamic diameter and size distribution of the particles dispersed in double distilled (DD) water were measured with a particle analyzer; model NANOPHOX (SympatecGmbH, Germany).

Electrokinetics properties (ζ-potential) of the formed particles were measured as a function of pH. The measurements were determined with potential analyzer model Zeta Potential WALLIS (Cordouan Technologies, France).

### Results

Characterization of the PMSF NPs:

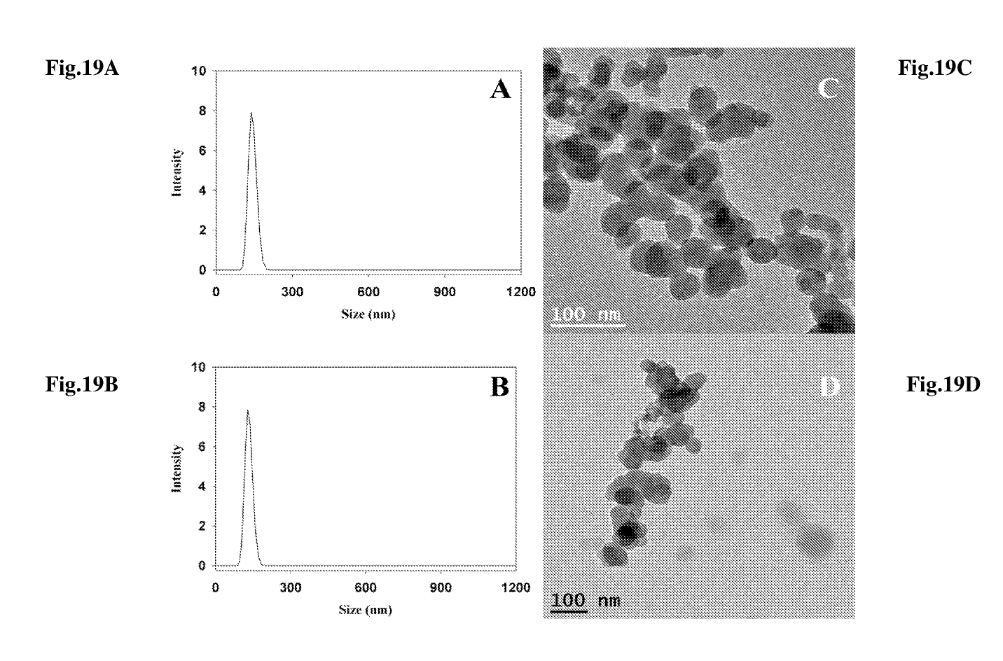

In exemplary procedures, PMSF NPs were prepared by dispersion co-polymerization in absence or presence of PVP as a stabilizer. The polymerization yield of PMSF NPs was found to be 75% and 52%, respectively. FIG. 19 presents a hydrodynamic size histograms and TEM images of the dried PMSF NPs. The dry diameter and size distribution of the NPs synthesized with or without PVP, are 40±9 nm (FIG. 19 C) and 58±25 nm (FIG. 19 D), respectively, and with hydrodynamic diameter and size distribution of 139±17 nm (FIG. 19A) and 129±17 nm (FIG. 19B), respectively. The dry diameter of the NPs is significantly smaller than the hydrodynamic diameter, probably, without being bound by a particular theory, due to the fact that the hydrodynamic diameter takes into account the solvent molecules adsorbed on the surface and within the NPs as well as the Brownian motion.

The NPs synthesized in absence of the stabilizer had a tendency of agglomeration in the aqueous continuous phase and exhibited a broader size distribution. Furthermore, when repeating the experiment, the obtained particles size was inconsistent, therefore the work continued only with the NPs prepared with the stabilizer, PVP.

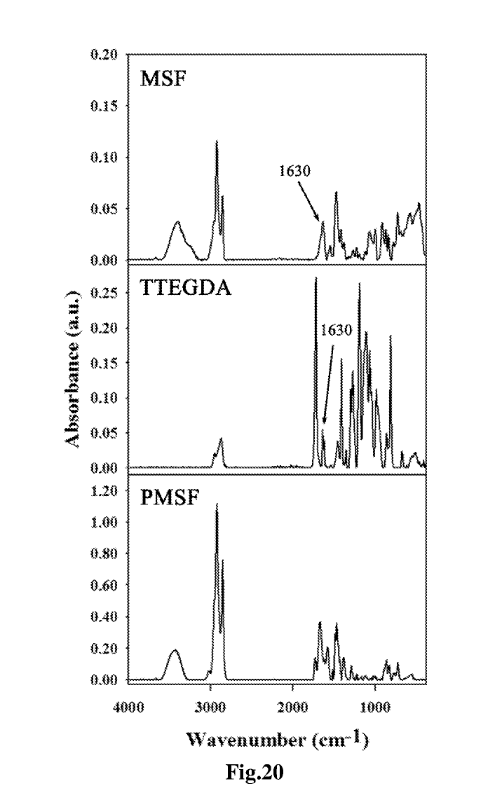

FIG. 20 illustrates the FTIR spectra of the MSF and TTEGDA monomers and the PMSF crosslinked NPs. The FTIR spectrum of the PMSF NPs shows typical absorption peaks corresponding to the peaks found in the FTIR spectra of the MSF and TTEGDA monomers. It should be noted that C═C stretching vibration band peak at 1630 cm⁻¹ in the monomers spectra disappears in the PMSF spectrum, due to the polymerization.

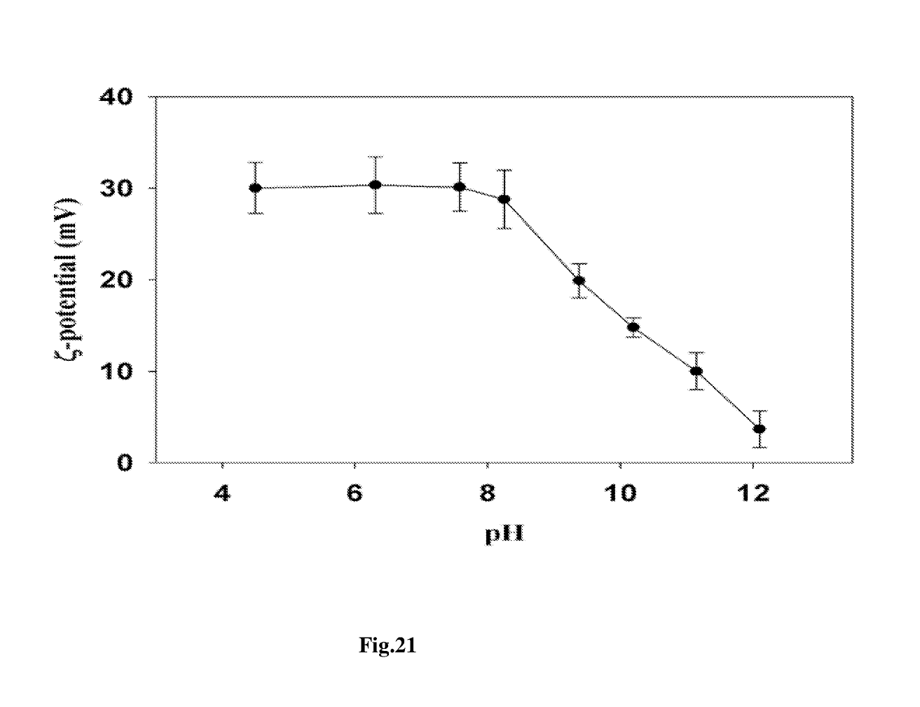

ζ-potential of the PMSF NPs dispersed in aqueous solution indicates their stability, a nanoparticle surface positively charge will create repulsion between NPs and thereby prevents aggregation. The change in the ζ-potential of the PMSF NPs as a function of pH is illustrated in FIG. 21. This figure shows that between pH 4.5-7.7 the ζ-potential of the PMSF NPs is not affected, about 30 mV, then increasing the pH up to 12.1 leads to sharp decrease in the potential, from 30.0 to 3.7 mV. This sharp decrease in the ζ-potential is probably attributed to the increasing concentration of non-charged groups. At a pH higher than 12.1, as the potential approaches the isoelectric point, the NPs are totally agglomerated.

Kinetics of the Polymerization:

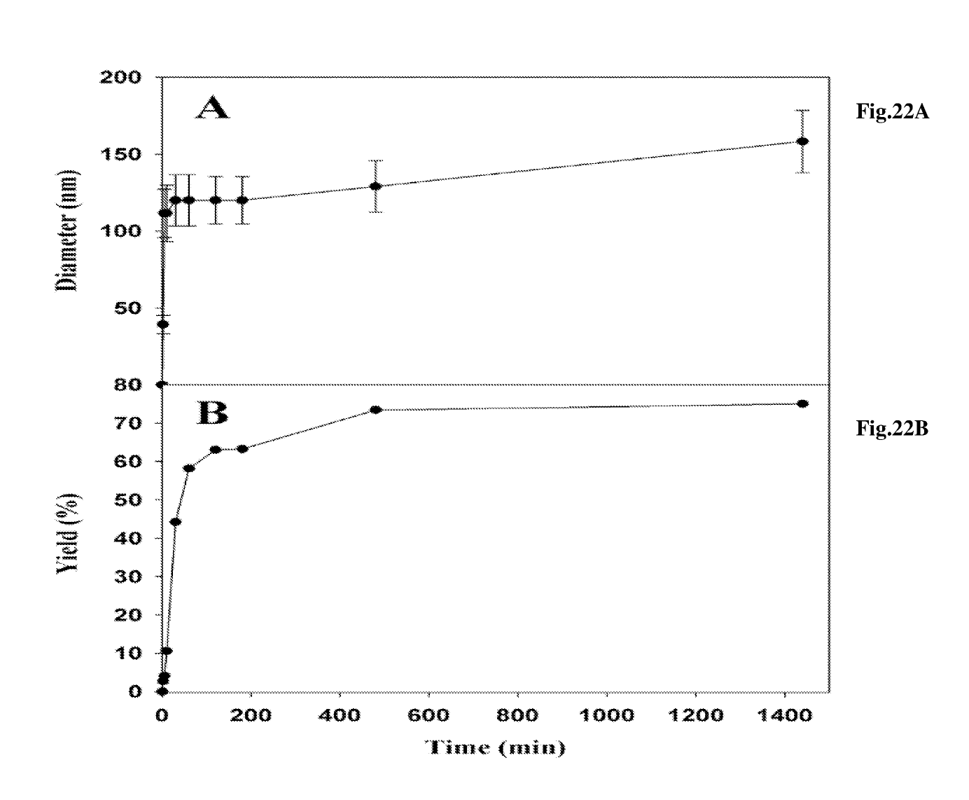

Kinetics of the polymerization of the PMSF NPs was evaluated (FIG. 22), by following the hydrodynamic size and the yield of the produced NPs. FIG. 22 (upper panel) illustrates the increase in the average hydrodynamic size of the formed PMSF NPs over time. During the first 30 minutes of the polymerization, there is a sharp increase in the average size of the produced NPs, followed by a much milder increase over the next few hours. For example, after 2, 30 and 480 min following the initiation of the polymerization process, the hydrodynamic size of the PMSF NPs increased from 39±6 to 120±17 and 129±17 nm, respectively. FIG. 22 (lower panel) further shows the change in the yield of the formed PMSF NPs, during the first 120 minutes of the polymerization, a sharp increase in the yield of the produced NPs is exhibited, followed by a much milder increase up to 75%. For example, 2, 30, 120 and 480 min following the initiation of the polymerization, the yield of the PMSF NPs increased from 3 to 44, 63 and 73%, respectively.

The kinetics of the polymerization displayed by following the change in particle diameter (FIG. 22, upper panel) is similar to the behavior of the yield (FIG. 22, lower panel), but there is one notable difference: after about 30 minutes the particles have almost reached their final diameter, while the yield of the produced PMSF NPs observed only after about 24 hours. Hence the NPs reach their final diameter while the monomer trapped in the NPs continues to polymerize.

### Example 8

### Effect of Various Polymerization Parameters on the Size and Size Distribution of PMSF NPs

Effect of the Total Monomers Concentration:

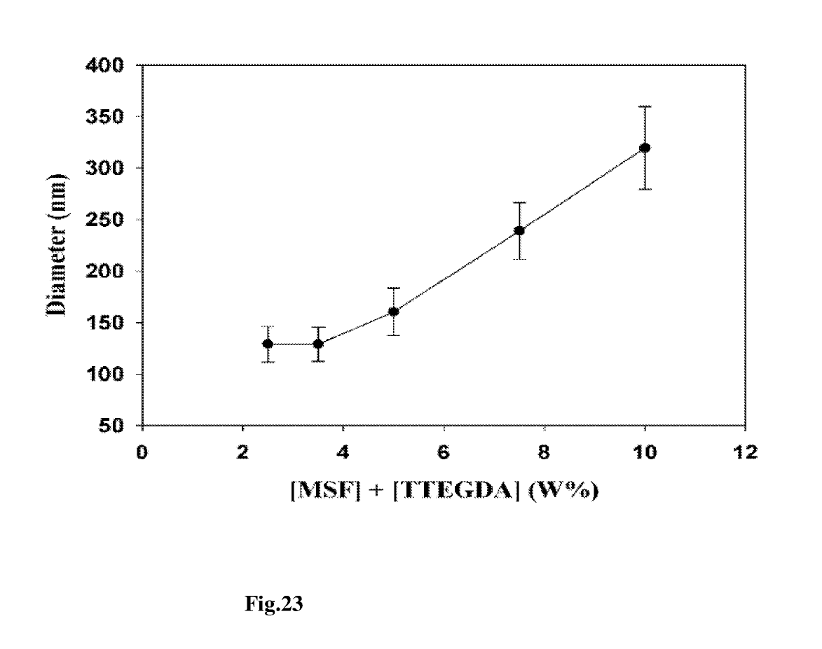

The effect of the total monomers concentration (while maintaining the weight ratio between the two monomers and the other polymerization parameters constant) on the size and size distribution of the formed PMSF NPs is illustrated in FIG. 23.

This figure illustrates that increasing the total monomers concentration increases the size and the size distribution of the formed PMSF NPs. For example, when increasing the total monomer concentration from 2.5 to 5 and 10% (w/v) the size and size distribution of the NPs increases from 129±17 to 160±23 and 320±40 nm, respectively. Increasing the concentration of the total monomers above 10% resulted in agglomeration of the NPs.

Effect of the Weight Ratio [TTEGDA]/[Total Monomers]:

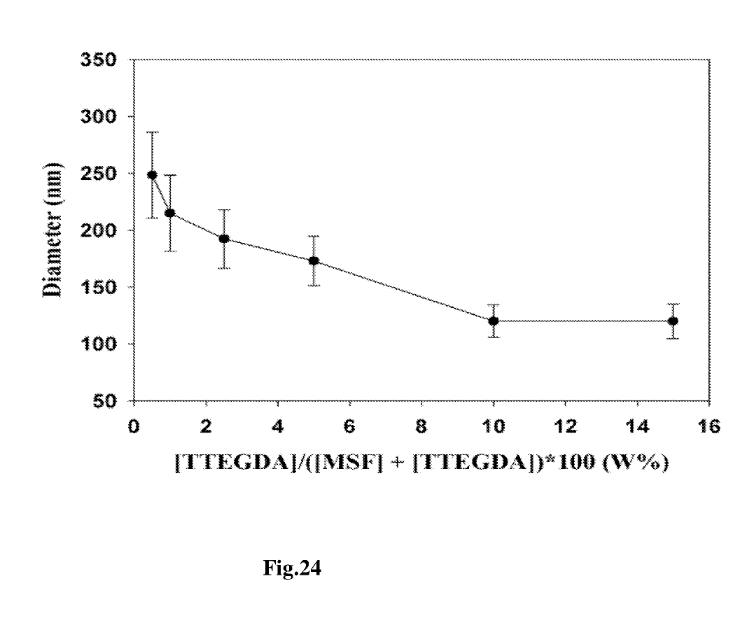

The effect of the weight ratio [TTEGDA]/([MSF]+[TTEGDA]) on the size and size distribution of the formed PMSF NPs, while the total monomers kept constant (5%) is shown in FIG. 24. The results indicate that when the weight ratio [TTEGDA]/[total monomers] increases the size and size distribution of the formed PMSF NPs decreases. For example, when increasing the ratio from 1 to 5 and 15% (w/w) the size and size distribution of the NPs decreases from 215±33 to 173±22 and 120±15 nm, respectively. This could be explained, without being bound by any particular theory, that by the fact that the higher the concentration of the crosslinking monomer, tightly packed crosslinked nuclei is formed and thus the growth of the nuclei by monomer swelling is reduced, resulting in smaller particles.

Effect of the Initiator Concentration:

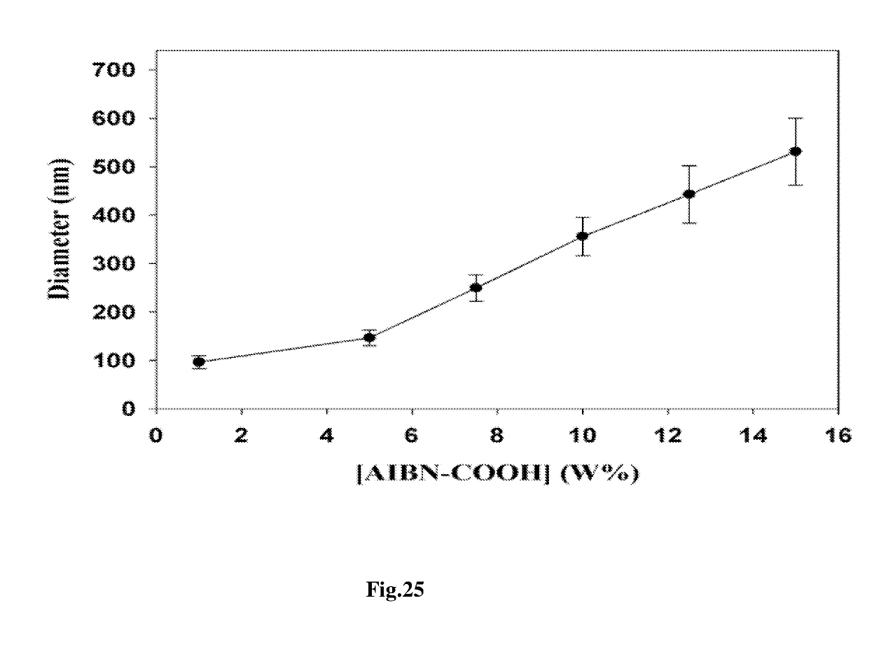

The effect of the initiator concentration on the size and size distribution of the formed PMSF NPs is shown in FIG. 25. When increasing the AIBN—COOH concentration the size and size distribution of the formed PMSF NPs increases. For example, when increasing the initiator concentration from 1 to 5 and 10% (w/w) the NPs size and size distribution increases from 97±13 to 147±17 and 356±39 nm, respectively.

Effect of Stabilizer Concentration:

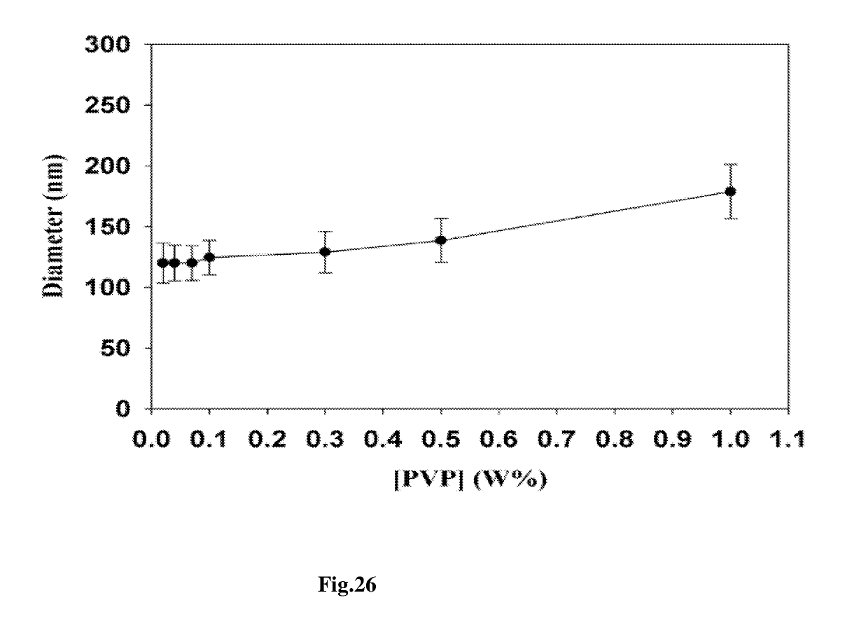

The effect of the PVP concentration on the size and size distribution of the formed PMSF NPs prepared by dispersion polymerization is illustrated in FIG. 26. A slight increase in the size of the formed NPs while increasing the stabilizer concentration was observed. For example, when increasing the PVP concentration from 0.07 to 0.5 and 1% (w/v) the NPs size increases from 120±14 to 139±18 and 179±22 nm, respectively. Many publications have reported the opposite behavior, an increase in the stabilizer concentration leads to the formation of smaller particles. [B. Peng and A. Imhof, Soft Matter, 2015, 11, 3589-3598. S. Shen, E. D. Sudol and M. S. Aasser, J. Polym. science Polym. Chem., 1993, 31, 1393-1402.]

Without being bound by any particular mechanism, it is assumed that increasing the stabilizer concentration can increase the continuous phase viscosity, this can slow down the oligoradicals diffusion and as a result there is interference with the termination stage of the polymerization process. On the other hand, the propagation stage of the polymerization process is not affected by the viscosity of the continuous phase, and small monomers molecules can still diffuse. This leads to an increase in the PMSF chains (concentration and size) and finally to an increase in the size of the NPs.

Antibacterial Properties of the PMSF NP:

The antibacterial activity of the PMSF NPs was evaluated as described hereinabove, by determining their MIC using E. coli and P. aeruginosa, two common bacterial pathogens representing Gram-negative bacteria and L. innocua and S. aureus bacteria which represent Gram-positive bacteria. The bacteria were exposed to a serial diluted concentration of PMSF in an aqueous dispersion and the MIC was found to be 0.93 mg/mL for E. coli and P. aeruginosa and 0.625 mg/mL for L. innocua and S. aureus as shown in Table 2 which represents the Minimum inhibitory concentration (MIC) of the PMSF NPs.

TABLE 2

| Bacterial Type | MIC (mg/mL) |
|---|---|
| E. coli, P. aeruginosa | 0.93 |
| L. innocua, S. aureus | 0.625 |

### Example 9

### Antibiofilm Activity of the PMSF NPs

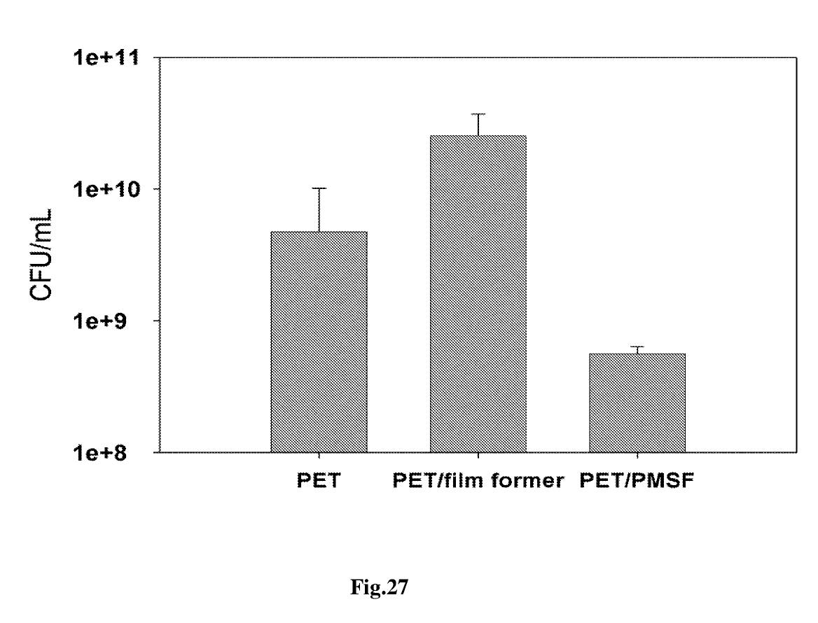

In light of the antibacterial activity exerted by the PMSF, the NPs were tested with respect to their ability to inhibit the biofilm formation of Listeria. Hence, PET films were coated with the NPs aqueous dispersion using a formulation with a ratio of 1:1 PMSF polymer to the film former (G-9/230). A knife (Mayer rod) of 6 (wet thickness) was used for the coating. As shown in FIG. 27, a significant reduction in the biofilm formation of Listeria was detected; 2 logs for the PET/PMSF films, in comparison to a film containing the film former only.

As noted hereinabove, migration may limit the use of additives in plastic especially for food packing applications, pharmaceutical and other hygienic products. Crosslinked NPs that are compatible with the PET film and the film former may overcome this disadvantage, due to their large spatial structure, which reduces their migration while maintaining the activity. Hence, using appropriate NPs as an additive to films will result in antibacterial properties and with decreased extractability and volatility. Indeed, no migration of the PMSF NPs to the continuous phase composed of 3% aqueous acetic acid or 95% ethanol were found.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A compound being in the form of Formula I: 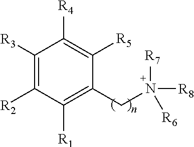
wherein each of R₁-R₈ represents a substituent,
wherein:
one to four substituents from R₁ to R₅, in each instance, comprise or are selected from the group consisting of hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, or is a fused ring;
R₆ to R₈, in each instance, comprises or is selected from the group consisting of hydrogen, alkyl, and cycloalkyl;
n is an integer having a value from 1 to 5, and
at least one of R₁ to R₅ is, independently, in the form represented by Formula II: 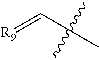
wherein R₉ is alkyl.

2. The compound of claim 1, wherein any one of (i) R₉ is CH₂, (ii) n is 1; (iii) R₃ is in the form represented by Formula II:

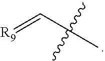

3. (canceled)

4. (canceled)

5. The compound of claim 1, being in the form of Formula Ib:

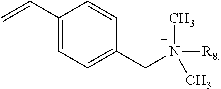

6. A polymer comprising a plurality of first monomeric unit, wherein said first monomeric unit is derived from the compound of claim 1.

7. The polymer of claim 6, wherein anyone of (i) said plurality of said first monomeric unit is in the form represented by Formula III: 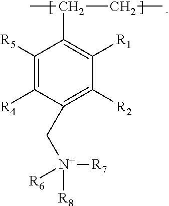
wherein m is an integer having a value from 2 to 3000;
and (ii) the polymer further comprising a plurality of a second monomeric unit, said second monomeric unit being derived from a cross-linking monomer.

8. (canceled)

9. The polymer of claim 7 comprising a plurality of polymeric backbone represented by Formula IIIc:
Yₐ—Z_{b};
wherein:
Y represents said first monomeric unit;
Z is said second monomeric unit, and
a and b are integers, each independently in each instance, represents the total numbers of Y and Z, respectively, in the polymeric backbone, and
wherein a and b, independently, have a value of 0, 1 or more;
optionally wherein said second monomeric unit is at least 1%, by weight.

10. (canceled)

11. A polymer comprising a plurality of first monomeric unit derived from a monomer, said first monomeric unit being in the form represented by Formula IV: 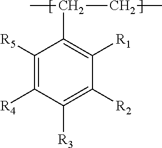
wherein each of R₁ to R₅ represents a substituent,
and wherein one to four substituents from R₁ to R₅, in each instance, comprise or are selected from the group consisting of hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, or is a fused ring, and
at least one of R₁ to R₅ is independently in the form represented by Formula V: 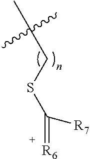
wherein n is an integer having a value from 1 to 5, and
R₆ and R₇ are each, independently selected from the group consisting of NH₂, and NHR₈, wherein R₈ is or alkyl.

12. The polymer of claim 11, wherein said first monomeric unit is selected from the group consisting of Formula IVb: 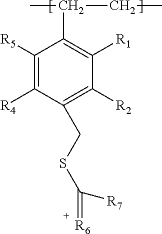
and Formula IVc:

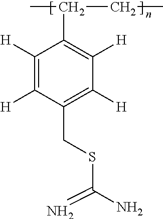

13. (canceled)

14. The polymer of claim 11, wherein any one of:
(i) the polymer further comprising a plurality of a second monomeric unit derived from a cross-linking monomer;
(ii) the polymer comprising a plurality of polymeric backbone, represented by Formula X:
X_{d}—Fₑ₋;
wherein:
X represents said first monomeric unit;
F represents second monomeric unit derived from a cross-linking monomer;
d and e are integers, each independently in each instance, represents the total numbers of X and F, respectively, in the polymeric backbone, and
wherein d and e, independently, have a value of 0, 1 or more; and
(iii) the polymer wherein said second monomeric unit is at least 1%, by weight;
optionally wherein said cross-linking monomer is selected from the group consisting of tetra(ethylene glycol) diacrylate, ethylene glycol dimethacrylate, and divinylbenzene.

15.-17. (canceled)

18. A composition-of-matter comprising the polymer of claim 6.

19. The composition-of-matter of claim 18, further comprising a stabilizer, optionally wherein said stabilizer is selected from the group consisting of polyvinylpyrrolidone (PVP), polysorbate Sodium dodecyl sulfate (SDS), sodium dodecyl benzene sulfate (SDBS), Poly(ethylene glycol)-block-poly(propylene glycol)-block-poly(ethylene glycol), and Polyethylene glycol sorbitan monolaurate (Tween 20).

20. (canceled)

21. The composition-of-matter of claim 16, being in the form of one or more particles.

22. The composition-of-matter of claim 21, wherein said one or more particles are microsized or nanosized.

23. (canceled)

24. The composition-of-matter of claim 18, wherein at least 80% of said particles are characterized by a size that varies within a range of less than 20%.

25. The composition-of-matter of claim 18, further comprising a substrate, wherein a plurality of said polymer is incorporated or coated in/on at least a portion of said substrate, optionally wherein said substrate is selected from (i) a substrate comprising or is made of a polymeric material is selected from the group consisting of polypropylene (PP), polycarbonate (PC), high-density polyethylene (HDPE), polyester (PE), polyethylene terephthalate (PET), polyvinyl chloride (PVC), and polytetrafluoroethylene (PTFE, Teflon®); (ii) a substrate comprising or is made of a wood, a metal, and glass; (iii) a substrate forming a part of an article.

26.-28. (canceled)

29. An article comprising the composition-of-matter of claim 18.

30. The article of claim 29, being selected from the group consisting of a medical device, fluidic device, water system device, tubing, an agricultural device, a package, a sealing article, a fuel container and a construction element.

31. A method of inhibiting or reducing a formation of load of a microorganism, and/or a formation of a biofilm or biofouling in and/or on an article, said microorganism being selected from the group consisting of: viruses, fungi, parasites, yeast, bacteria, and protozoa.

32. A process of producing a particle comprising a polymer, said process comprising the step of free radical homopolymerization or copolymerization of monomers of the compound of claim 1 in the presence of (a) a free radical initiator; (b) a cross-linking monomer and (c) a stabilizer, thereby making the particle.

33. The process of claim 32, wherein
(i) a reaction mixture for making the particle comprises: 1-20% by weight of said monomers of the compound;
1-20% by weight of said cross-linking monomers;
0.2-20% by weight of a free radical initiator; and
0.1-20% by weight of said stabilizer;
(ii) wherein said initiator is selected from the group consisting of: azo-bis-isobutyronitrile (AIBN), AIBNCO₂H, potassium persulfate (PPS), benzoyl peroxide, and H₂O₂;
(iii) wherein said cross-linking monomer is selected from the group consisting of tetra(ethylene glycol) diacrylate, ethylene glycol dimethacrylate, and divinylbenzene;
or any combination thereof.

34.-35. (canceled)
